SEQUENCE LISTING (40 sequences)

SEQ ID NO: 1 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 2 - protein, 2366 aa, Clostridium difficile
MSLVNRKQLEKMANVRFRVQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFYRKRMEIIYDKQKNFINYYKTQREENPDLIIDDIVKIYLSNEYSKDIDELNSYIEESLNKVTENSGNDVRNFEEFKGGESFKLYEQELVERWNLAAASDILRISALKEVGGVYLDVDMLPGIQPDLFESIEKPSSVTVDFWEMVKLEAIMKYKEYIPGYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKKNYFEGSLGEDDNLDFSQNTVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIDTQVVEGRIEEAKSLTSDSINYIKNEFKLIESISDALYDLKQQNELEESHFISFEDILETDEGFSIRFIDKETGESIFVETEKAIFSEYANHITEEISKIKGTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELILRDKATKVVDYFSHISLAESEGAFTSLDDKIMMPQDDLVISEIDFNNNSITLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPVITTEYIREKLSYSFYGSGGTYALSLSQYNMNINIELNENDTWVIDVDNVVRDVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDKGKENGFINCSTKEGLFVSELSDVVLISKVYMDNSKPLFGYCSNDLKDVKVITKDDVIILTGYYLKDDIKISLSFTIQDENTIKLNGVYLDENGVAEILKFMNKKGSTNTSDSLMSFLESMNIKSIFINSLQSNTKLILDTNFIISGTTSIGQFEFICDKDNNIQPYFIKFNTLETKYTLYVGNRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 3 - protein, 716 aa, Clostridium difficile
NRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 4 - protein, 2710 aa, Clostridium difficile
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALTSYKIINGKHFYFNNDGVMQLGVFKGPDGFEYFAPANTQNNNIEGQAIVYQSKFLTLNGKKYYFDNNSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 5 - protein, 2710 aa, Clostridium difficile
MSLISKEELIKLAYSIRPRENEYKTILTNLDEYNKLTTNNNENKYLQLKKLNESIDVFMNKYKNSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETLLESYRTNSLRKINSNHGIDIRANSLFTEQELLNIYSQELLNRGNLAAASDIVRLLALKNFGGVYLDVDMLPGIHSDLFKTIPRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKDSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKEYGPLKTEDDKILVPIDDLVISEIDFNNNSIKLGTCNILAMEGGSGHTVTGNIDHFFSSPYISSHIPSLSVYSAIGIKTENLDFSKKIMMLPNAPSRVFWWETGAVPGLRSLENNGTKLLDSIRDLYPGKFYWRFYAFFDYAITTLKPVYEDTNTKIKLDKDTRNFIMPTITTDEIRNKLSYSFDGAGGTYSLLLSSYPISMNINLSKDDLWIFNIDNEVREISIENGTIKKGNLIEDVLSKIDINKNKLIIGNQTIDFSGDIDNKDRYIFLTCELDDKISLIIEINLVAKSYSLLLSGDKNYLISNLSNTIEKINTLGLDSKNIAYNYTDESNNKYFGAISKTSQKSIIHYKKDSKNILEFYNGSTLEFNSKDFIAEDINVFMKDDINTITGKYYVDNNTDKSIDFSISLVSKNQVKVNGLYLNESVYSSYLDFVKNSDGHHNTSNFMNLFLNNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTSIASTGYTIINGKYFYFNTDGIMQIGVFKVPNGFEYFAPANTHNNNIEGQAILYQNKFLTLNGKKYYFGSDSKAITGWQTIDGKKYYFNPNNAIAATHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDSKKYYFNLNTAVAVTGWQTIDGEKYYFNLNTAEAATGWQTIDGKRYYFNTNTYIASTGYTIINGKHFYFNTDGIMQIGVFKGPDGFEYFAPANTHNNNIEGQAILYQNKFLTLNGKKYYFGSDSKAVTGLRTIDGKKYYFNTNTAVAVTGWQTINGKKYYFNTNTYIASTGYTIISGKHFYFNTDGIMQIGVFKGPDGFEYFAPANTDANNIEGQAIRYQNRFLYLHDNIYYFGNDSKAATGWATIDGNRYYFEPNTAMGANGYKTIDNKNFYFRNGLPQIGVFKGPNGFEYFAPANTDANNIDGQAIRYQNRFLHLLGKIYYFGNNSKAVTGWQTINSKVYYFMPDTAMAAAGGLFEIDGVIYFFGVDGVKAPGIYG

SEQ ID NO: 6 - protein, 879 aa, Clostridium difficile
GLININNSLFYFDPIESNLVTGWQTINGKKYYFDINTGAASTSYKIINGKHFYFNNNGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSGSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTSIASTGYTIINGKYFYFNTDGIMQIGVFKVPNGFEYFAPANTHNNNIEGQAILYQNKFLTLNGKKYYFGSDSKAITGWQTIDGKKYYFNPNNAIAATHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDSKKYYFNLNTAVAVTGWQTIDGEKYYFNLNTAEAATGWQTIDGKRYYFNTNTYIASTGYTIINGKHFYFNTDGIMQIGVFKGPDGFEYFAPANTHNNNIEGQAILYQNKFLTLNGKKYYFGSDSKAVTGLRTIDGKKYYFNTNTAVAVTGWQTINGKKYYFNTNTYIASTGYTIISGKHFYFNTDGIMQIGVFKGPDGFEYFAPANTDANNIEGQAIRYQNRFLYLHDNIYYFGNDSKAATGWATIDGNRYYFEPNTAMGANGYKTIDNKNFYFRNGLPQIGVFKGPNGFEYFAPANTDANNIDGQAIRYQNRFLHLLGKIYYFGNNSKAVTGWQTINSKVYYFMPDTAMAAAGGLFEIDGVIYFFGVDGVKAPGIYG

SEQ ID NO: 7 - protein, 18 aa, Clostridium difficile
SPNIYTDEINITPVYETN

SEQ ID NO: 8 - protein, 16 aa, Clostridium difficile
YPEVIVLDANYINEKI

SEQ ID NO: 9 - protein, 15 aa, Clostridium difficile
TVGDDKYYFNPINGG

SEQ ID NO: 10 - protein, 18 aa, Clostridium difficile
ASIGETIIDDKNYYFNQS

SEQ ID NO: 11 - protein, 16 aa, Clostridium difficile
EDGFKYFAPANTLDEN

SEQ ID NO: 12 - protein, 12 aa, Clostridium difficile
PANTLDENLEGE

SEQ ID NO: 13 - protein, 12 aa, Clostridium difficile
AIDFTGKLIIDE

SEQ ID NO: 14 - protein, 14 aa, Clostridium difficile
NIYYFDDNYRGAVE

SEQ ID NO: 15 - protein, 12 aa, Clostridium difficile
HYFSPETGKAFK

SEQ ID NO: 16 - protein, 13 aa, Clostridium difficile
IGDYKYFNSDGVM

SEQ ID NO: 17 - protein, 22 aa, Clostridium difficile
HFYFAENGEMQIGVFNTEDGFK

SEQ ID NO: 18 - protein, 16 aa, Clostridium difficile
INDGQYYFNDDGIMQV

SEQ ID NO: 19 - protein, 16 aa, Clostridium difficile
YKYFAPANTVNDNIYG

SEQ ID NO: 20 - protein, 14 aa, Clostridium difficile
ESDKYYFNPETKKA

SEQ ID NO: 21 - protein, 19 aa, Clostridium difficile
NNNYYFNENGEMQFGYINI

SEQ ID NO: 22 - protein, 16 aa, Clostridium difficile
QNTLDENFEGESINYT

SEQ ID NO: 23 - protein, 18 aa, Clostridium difficile
SPNIYTDEINITPIYEAN

SEQ ID NO: 24 - protein, 16 aa, Clostridium difficile
YPEVIVLDTNYISEKI

SEQ ID NO: 25 - protein, 15 aa, Clostridium difficile
TIGDDKYYFNPDNGG

SEQ ID NO: 26 - protein, 18 aa, Clostridium difficile
ASVGETIIDGKNYYFSQN

SEQ ID NO: 27 - protein, 16 aa, Clostridium difficile
EDGFKYFAPADTLDEN

SEQ ID NO: 28 - protein, 12 aa, Clostridium difficile
PADTLDENLEGE

SEQ ID NO: 29 - protein, 12 aa, Clostridium difficile
AIDFTGKLTIDE

SEQ ID NO: 30 - protein, 14 aa, Clostridium difficile
NVYYFGDNYRAAIE

SEQ ID NO: 31 - protein, 12 aa, Clostridium difficile
YYFSTDTGRAFK

SEQ ID NO: 32 - protein, 14 aa, Clostridium difficile
IGDDKFYFNSDGIM

SEQ ID NO: 33 - protein, 22 aa, Clostridium difficile
YFYFAENGEMQIGVFNTADGFK

SEQ ID NO: 34 - protein, 16 aa, Clostridium difficile
INDGKYYFNDSGIMQI

SEQ ID NO: 35 - protein, 15 aa, Clostridium difficile
YKYFAPANTVNDNIY

SEQ ID NO: 36 - protein, 14 aa, Clostridium difficile
ESDKYYFDPETKKA

SEQ ID NO: 37 - protein, 19 aa, Clostridium difficile
DNHYYFNEDGIMQYGYLNI

SEQ ID NO: 38 - protein, 16 aa, Clostridium difficile
QNTLDENFEGESINYT

SEQ ID NO: 39 - protein, 190 aa, Clostridium difficile
DDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIA

SEQ ID NO: 40 - protein, 190 aa, Clostridium difficile
DENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIA

(19) United States
(12) Patent Application Publication
(10) Pub. No.: US 2021/0369831 A1
(43) Pub. Date: Dec. 2, 2021

(54) CLOSTRIDIUM DIFFICILE MULTI-COMPONENT VACCINE

(71) Applicant: Matrivax, Inc., Boston, MA (US)

(72) Inventors: Kevin Killeen, Boston, MA (US); Thomas Griffin, Boston, MA (US)

(21) Appl. No.: 17/321,719

(22) Filed: May 17, 2021

Related U.S. Application Data
(63) Continuation of application No. PCT/US2019/061793, filed on Nov. 15, 2019.
(60) Provisional application No. 62/768,220, filed on Nov. 16, 2018.

(51) Int. Cl. A61K 39/08; A61K 45/06

(52) U.S. Cl. CPC A61K 39/08; A61K 2039/521; A61K 45/06

(57) ABSTRACT

Immunogenic compositions for combating C. difficile infection are disclosed comprising an admixture of at least two components (a) and (b), where
component (a) comprises inactivated cells of at least one strain of C. difficile, or cell surface extracts (CSE) from one or more strains of C. difficile bacteria; and
component (b) comprises at least one toxoid or a non-toxic, immunogenic polypeptide fragment of a C. difficile Toxin A or Toxin B. Administration of the immunogenic composition is effective to elicit an immune response in a subject immunized with said composition to produce antibodies reactive with at least one C. difficile strain and at least one C. difficile toxin.

### RELATED APPLICATIONS AND INCORPORATION BY REFERENCE

This application claims priority to U.S. provisional patent application Ser. No. 62/768,220, filed on Nov. 16, 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention is directed to immunogenic compositions, methods of making vaccines, and methods of vaccine administration. Specifically, the invention relates to Clostridium difficile vaccines comprising, in admixture, (a) inactivated C. difficile whole cells or cell extracts and (b) one or more polypeptides comprising a toxoid or a non-toxic, immunogenic polypeptide fragment of C. difficile Toxin A or Toxin B.

### BACKGROUND OF THE INVENTION

Clostridium difficile, a multi-drug resistant, spore-forming bacterium on the CDC 2013 Urgent Threats list (Antibiotic Resistance Threats in the United States, 2013 (AR Threats Report) https://www.cdc.gov/drugresistance/biggest_threats.html), is a common cause of healthcare-acquired infections occurring principally in older adults taking antibiotic regimens or experiencing prolonged hospital stays. Ironically, although C. difficile infections (CDI) are not yet significantly resistant to antibiotics, most infections are directly related to antibiotic therapy. Thus, CDI is commonly termed antibiotic associated diarrhea (AAD).

Clostridium difficile is recognized as the most important single identifiable cause of nosocomial antibiotic-associated diarrhea and colitis, and CDI has now also emerged in the community in populations previously considered low risk, such as healthy peripartum women, children, antibiotic naïve patients, and those with minimal or no recent healthcare exposure (Centers for Disease Control and Prevention (CDC), Severe Clostridium difficile-associated disease in populations previously at low risk-four states, MMWR Morb Mortal Wkly Rep., 54: 1201-1205 (2005)).

CDI is responsible for 10 to 25% of all cases of antibiotic-associated diarrhea and for almost all cases of pseudomembranous colitis (Bartlett, J. G., Clin. Infect. Dis., 18(Suppl. 4): S265-S272 (1994)). In recent years, a dramatic increase in the incidence of C. difficile diarrhea has been observed, noted by a marked increase in incidence and severity (DePestel, D. et al., J. Pharm. Pract., 26(5): 464-475 (2013)). Paralleling this increased prevalence there has also been a corresponding increase in morbidity and mortality associated with CDI, which has coincided with the emergence and rapid spread of a previously rare strain, designated synonymously as polymerase chain reaction (PCR) ribotype 027, North American Pulse-field type 1 (NAP1), or restriction endonuclease analysis (REA) type BI, heretofore referred to as ribotype 027. (Zilberberg M D, Emerg. Infect. Dis.; 14:1756-1758 (2008)).

A 2015 CDC study found that there were nearly half a million cases of CDI in the United States per year that led to 15,000 deaths. The average cost for a single inpatient case of CDI is >$35,000 and the estimated annual cost burden for the healthcare system exceeds $3 billion.

Nosocomial outbreaks are frequent in hospitals and nursing homes, are difficult to control, and may occur even after a hospital ward has been closed and decontaminated (Bender, B. S., et al.; Lancet ii:11-13 (1986), 31). In tertiary-care hospitals, up to 37% of patients become infected, and 7.8% become ill (Samore, M. H., et al., Clin. Infect. Dis. 18:181-187 (1994)). Although most patients with C. difficile diarrhea respond to therapy, relapses may occur at a frequency of up to 55% (Bartlett, J. G., Clin. Infect. Dis., 18(Suppl. 4): S265-S272 (1994)).

C. difficile exerts its effects on the gastrointestinal (GI) tract by releasing two toxins that can bind to and damage intestinal epithelium. Toxins A (an enterotoxin) and B (a cytotoxin) contribute differently to the pathophysiology of CDI. Toxin A is associated with the secretion of fluid and generalized inflammation in the GI tract. Toxin B is considered the main determinant of virulence in recurrent CDI and is associated with more severe damage to the colon (Censers for Disease Control and Prevention Healthcare associated infections; https://www.cdc.gov/hai/organisms/cdiff/cdiff_clinicians.html).

Recent focus on C. difficile Toxin B (TcdB) has revealed two isoforms, the Toxin B from historical or non-hypervirulent strains (“Toxin B_{HIST}”) such as VPI 10463 (“TcdB1”, SEQ ID NO:1) and the Toxin B from hypervirulent strains (“Toxin B_{HV}”) of C. difficile such as BI/NAP1/027 (“TcdB2”, SEQ ID NO:2). In hypervirulent strains, Toxin B_{HV} is approximately 10-fold more cytotoxic in cell-based assays and at least 4-fold more lethal in a murine toxin challenge model than Toxin B from classical strains (Toxin B_{HIST}). The increased toxicity of Toxin B_{HV} is due to differences in the amino acid sequence of the C-terminal region (i.e., in the span of amino acids from position 1651 to the C-terminal position 2366).

At present, methods for primary and secondary prevention of CDI are controversial. Many forms of prophylaxis have been proposed, including probiotics and antibiotics, though none are recommended by the Infectious Disease Society of America (IDSA). The only preventative measures currently included in the IDSA guidelines are antimicrobial stewardship and the maintenance of clean, disinfected surfaces to promote sanitary conditions (Kociolek L K, et al., Nat Rev Gastroenterol Hepatol, 13 (3): 150-60 (2016)).

The potential severity of and damage caused by CDI in combination with its rising incidence renders the subject of prophylaxis a pressing public health concern. Previous research into various toxoid vaccines suggests their promise as preventative measures. Three investigational vaccines have been evaluated in Phase 2/3 clinical trials. Vaccine candidates in advanced clinical development target Toxin A and Toxin B_{HIST}. Recently, Sanofi discontinued development of its C. difficile toxoid A and toxoid B combination vaccine, indicating a toxoid-only prophylactic approach is not sufficient to prevent CDI recurrent disease. Pfizer and Valneva continue to advance their respective toxoid-based vaccine programs, which are based on Toxin A and Toxin B_{HIST} toxoids. The FDA has approved Merck's bezlotoxumab (Zinplava™), a monoclonal antibody targeting C. difficile Toxin B, for use in combination with antibiotic therapy for treatment of patients with CDI for the prevention of recurrent CDI, however Zinplava™ is only partially effective in ameliorating the symptoms associated with CDI, is less effective against hypervirulent C. difficile strains, and a decrease in CDI recurrence of only about 40% was observed in patients with CDI.

In view of the increasing incidence of CDI and the absence from the market of an effective vaccine for prevention of CDI, the need remains to discover an effective prophylactic approach for raising an immune response that will be protective against C. difficile infection. Furthermore, there is a need for improved therapeutic vaccines and immunotherapies for treatment of CDI.

### SUMMARY OF THE INVENTION

The present invention provides a combination vaccine approach to combat C. difficile infection. A vaccine composition as described herein is designed to target both C. difficile bacteria as the infectious agent and the main C. difficile enterotoxins as the most virulent products produced by C. difficile infection.

The present invention relates to an immunogenic composition comprising:

(a) inactivated whole cells of one or more strains of C. difficile bacteria or cell surface extracts (CSE) from one or more strains of C. difficile bacteria, and

(b) at least one polypeptide comprising a toxoid or a non-toxic, immunogenic polypeptide fragment of a C. difficile Toxin A or Toxin B, wherein said composition, when administered to a mammalian subject, is effective to elicit production of protective antibodies that bind to a wild type C. difficile and elicit protective antibodies that bind to at least one C. difficile toxin.

In particular embodiments, the immunogenic composition of the invention will include inactivated cells or a cell surface extract (CSE) from one or more C. difficile strains. For example, an immunogenic composition according to the invention may include inactivated C. difficile cells or a CSE from one or more C. difficile strains. In certain embodiments, the C. difficile strain comprises C. difficile ribotype 001, 003, 027, 106, 012, 014, 036, or 078.

In one embodiment, an immunogenic composition of the present invention comprises inactivated cells or cell surface extracts of two C. difficile strains. For example, component (a) of the immunogenic composition described herein may include inactivated cells of C. difficile strain VPI 10463 and inactivated cells of a C. difficile BI/NAP1/027 strain.

For the whole cell component (a) of the immunogenic compositions described herein, the C. difficile cells selected will be inactivated (i.e., killed), e.g., by heat treatment, UV or gamma radiation, or chemical treatment with reagents such as formaldehyde or formalin, alcohols (such as ethanol, isopropyl alcohol, phenol, tricresol, and the like), acetone, thimerosal, or antibiotics (such as β-propiolactone (BPL)). In specific embodiments, the C. difficile cells for component (a) are inactivated by heat treatment for a sufficient period to kill substantially all the cells. In further embodiments, the heat treatment is performed at above 55° C., for example at 56° for one hour, at 80° C. for thirty minutes, or at 100° C. for fifteen minutes.

In alternative embodiments, the whole cell component (a) of the immunogenic composition is prepared by treating C. difficile cells with formalin (i.e., a solution of formaldehyde, typically 37%, in water). Formalin treatment may suitably be carried out using 1%-5% v/v formalin in a cell suspension.

In alternative embodiments, the whole cell component (a) comprises a cell surface extract (CSE) from one or more C. difficile strains. In certain embodiments, the CSE is prepared from a suspension of C. difficile cells in deoxycholate. In certain embodiments, the CSE comprises one or more proteins set forth in Table 1. In certain embodiments, the CSE comprises one or more of CbpA, GroEL, CD3246, CD2381, CD0873, Dif51, Dif130, Dif192, Dif208, Dif208A, Dif232, CDT. In certain embodiments, an extract from C. difficile cells is supplemented by the addition of one or more of CbpA, GroEL, CD3246, CD2381, CD0873, Dif51, Dif130, Dif192, Dif208, Dif208A, Dif232, CDT, and the proteins set forth in Table 1. The supplemental proteins can be purified from C. difficile or recombinant micro-organisms.

For the C. difficile polypeptide component (b) of the immunogenic compositions described herein, the selected polypeptide may be a full-length C. difficile Toxin A and/or Toxin B inactivated to a toxoid form, e.g., by treatment with formaldehyde, or said polypeptide may be a non-toxic fragment of Toxin A and/or Toxin B that retains immunogenicity. Particular mention is made of polypeptides prepared from the C-terminal domain of C. difficile Toxin B that contains combined repetitive oligopeptides or CROPs. Particular embodiments will include one or more C. difficile polypeptide comprising all or a portion of the C-terminal 716 amino acids of C. difficile Toxin B. In a particular embodiment, the polypeptide component (b) of the immunogenic composition according to the invention comprises TcdB₁₆₅₁₋₂₃₆₆ of a hypervirulent BI/NAP1/027 strain of C. difficile. In another embodiment, the composition comprises a TcdB fragment having at least 50% identity to SEQ ID NO:7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In another embodiment, the non-toxic polypeptide component comprises a TcdA or TcdB protein or fragment thereof that is detoxified by mutation of one or more of its glucosyltransferase, serine protease, or delivery domain. In another embodiment, the non-toxic polypeptide fragment of C. difficile Toxin A binds to actoxumab. In another embodiment, non-toxic polypeptide fragment of C. difficile Toxin B binds to bezlotoxumab.

In additional embodiments, the C. difficile toxin polypeptide component (b) of the immunogenic composition comprises a polypeptide fragment of Toxin A or Toxin B or both Toxin A and B from one or more C. difficile strains. In particular embodiments, the C. difficile non-toxic polypeptide fragment is from one or more strains selected from the group consisting of C. difficile ribotypes 001, 003, 027, 106, 012, 014, 036, 078 and others. In further embodiments, the C. difficile toxin polypeptide is from a hypervirulent strain of C. difficile. In a specific embodiment, the C. difficile polypeptide fragment is from Toxin B of a C. difficile BI/NAP1/027 strain. In a specific embodiment, the non-toxic fragment of Toxin B has the amino acid sequence of SEQ ID NO:3.

In other embodiments, the C. difficile toxin polypeptide component (b) of the immunogenic compositions described herein may employ a C. difficile toxoid of Toxin A or Toxin B, that is, a full-length toxin protein that has been rendered non-toxic but retains immunogenic properties sufficient to induce an antibody response in an inoculated mammalian subject that recognizes a wild type toxin corresponding to the toxoid employed. Toxin A or Toxin B of C. difficile may be made non-toxic by any suitable means, such as by treatment with formaldehyde.

As used herein, the term “wild type” is used to indicate a C. difficile strain, gene, or characteristic found or observed among C. difficile in natural conditions.

In particular embodiments, the polypeptide component (b) may comprise more than one immunogenic C. difficile polypeptide. In other embodiments the component (b) will comprise a mixture of two or more C. difficile polypeptides. A suitable component (b) will comprise at least one of a C. difficile Toxin A toxoid, a C. difficile Toxin B toxoid, a non-toxic fragment of a C. difficile Toxin A, a non-toxic fragment of a C. difficile Toxin B, or combinations of any or all of the foregoing polypeptides, from one strain of C. difficile or multiple strains.

In an embodiment, the inactivated cells component (a) of the immunogenic composition comprises a naturally occurring (wild type) C. difficile strain or cell surface extracts (CSE) from such a strain. In another embodiment, the inactivated cells component (a) comprises inactivated cells of a non-toxinogenic strain of C. difficile, a sporulation deficient strain of C. difficile, or a strain that is both non-toxinogenic and sporulation deficient or cell surface extracts (CSE) from these strains. In another embodiment, the inactivated cells component or cell surface extract (CSE) (a) is from C. difficile cells from a BI/NAP1/027 strain, and the non-toxic polypeptide fragment component (b) of the immunogenic composition comprises an immunogenic portion of Toxin B from a C. difficile BI/NAP1/027 strain. In particular embodiments, the non-toxic Toxin B fragment has the amino acid sequence of SEQ ID NO:3.

In a further embodiment, the immunogenic composition of the invention also comprises an additional component (c), an adjuvant. In particular embodiments, an adjuvant is selected from the group of alum, mineral oil, vegetable oils, aluminum hydroxide, Freund's incomplete adjuvant, and TLR agonists (such as CpG oligonucleotides).

In other embodiments, the present invention relates to a method of making an immunogenic composition effective for eliciting a protective immune response producing antibodies reactive with one or more strains of C. difficile and antibodies reactive with one or more C. difficile toxins, in particular Toxin A or Toxin B proteins, the method comprising:
(1) admixing a first component (a) comprising inactivated cells, or cell surface extracts (CSE), of at least one strain of C. difficile in an amount effective to elicit an immune response in a mammalian subject immunized with said first component to produce antibodies reactive with at least one strain of C. difficile with a second component (b) comprising at least one polypeptide comprising a toxoid or a non-toxic, immunogenic polypeptide fragment of a C. difficile Toxin A or Toxin B in an amount effective to elicit an immune response in a mammalian subject immunized with said second component to produce antibodies reactive with at least one C. difficile Toxin A or Toxin B; and
(2) formulating said admixture of step (1) for administration to a mammalian subject susceptible to C. difficile infection.

It is desirable that the toxoid and/or non-toxic polypeptide Toxin A or Toxin B fragment employed will be effective to elicit production of antibodies that neutralize Toxin A or Toxin B in an immunized subject.

The present invention further provides a method of eliciting an immune response in a mammalian subject against C. difficile, said method comprising administering to said subject an amount of an immunogenic composition comprising (a) inactivated cells of at least one strain of C. difficile bacteria, or cell surface extracts (CSE), and (b) at least one polypeptide comprising a toxoid or a non-toxic, immunogenic polypeptide fragment of a C. difficile Toxin A or Toxin B.

The present invention also provides a method of prophylactically reducing the pathological effects of or preventing C. difficile infection in a subject, said method comprising administering to said subject a composition comprising (a) inactivated cells, or cell surface extracts (CSE), of at least one strain of C. difficile bacteria, and (b) at least one polypeptide comprising a toxoid or a non-toxic, immunogenic polypeptide fragment of a C. difficile Toxin A or Toxin B.

Additional embodiments and advantages of the compositions and methods according to the present invention are described below in detail.

### BRIEF DESCRIPTION OF THE DRAWINGS

FIG. 1 is a graph depicting the percentage change in body weight of mice relative to their starting weight prior to infection. Weight loss is a surrogate marker of morbidity of C. difficile infection in mice. Groups of mice (n=5) were immunized intraperitoneally three times at biweekly intervals with alum-adjuvanted, inactivated BI/NAP1/027 cells (“whole cell vaccine”, abbreviated WCV in the figure). Additional treatment groups were immunized with a combination of WCV and an immunogenic polypeptide comprised of a TcdB2 C-terminal fragment (BI/NAP1/027 Toxin B amino acids 1651-2366, designated “CROP” in the figure). About 2.5 weeks after the third immunization, a fourth immunization of 10⁹ WCV cells was administered to mice intraperitoneally, with or without CROP in addition. About 2.5 weeks after the fourth immunization, mice underwent an antibiotic treatment regimen for 7 days followed by intragastric administration of 10⁷ C. difficile spores per dose. Body weight measurements and fecal pellets from each individual animal were taken at the indicated intervals for the remainder of the study for twenty days post-infection. The plotted points are the geometric means of the percentage of weight relative to the weight on Day 0 of the five mice in each experimental group.

FIG. 2 is a graph presenting survival curves and geometric time to death of mice challenged with virulent C. difficile. Mice (n=10) were immunized intraperitoneally three times at biweekly intervals with alum-adjuvanted toxoid B_{HV} or CROPB_{HV}. Two weeks after the third immunization, mice were challenged with 100 ng of Toxin B_{HV} and monitored for survival. In this experiment, there were two naïve groups, one the was challenged with Toxin B_{HV} and another that was not challenged. The data demonstrate that immunization with CROPB_{HV} conferred complete protection against Toxin B_{HV} challenge while immunization with Toxoid B_{HV} conferred partial protection, 80%, against toxin challenge.

### DETAILED DESCRIPTION OF THE INVENTION

Vaccines and monoclonal antibody therapies targeting only the toxins produced by C. difficile can neutralize the cause of disease symptoms associated with CDI but do not target the bacteria themselves, which are the source of enhanced toxin production during infection and are a reservoir for re-infection and transmission. A novel approach disclosed herein for a protective C. difficile vaccine is to target both bacterial surface antigens presented on C. difficile cells and bacterial toxins produced by C. difficile cells. The combination vaccine concept disclosed herein is aimed at preventing CDI (and recurrence of CDI) in patients at high risk for contracting CDI which include the elderly, adults with planned hospitalization, Long Term Care Facility residents and patients with co-morbidity requiring prolonged use of antibiotics.

The two-component immunogenic compositions disclosed herein are designed to elicit a protective immune response that targets both C. difficile bacterial surface antigens and C. difficile Toxin A and/or Toxin B. Thus, vaccines of the invention have the capacity to be effective both prophylactically and therapeutically.

Inactivated bacterial whole cells have proven efficacy as immunogens in the whole cell pertussis vaccine and with live attenuated tuberculosis, typhoid fever, and cholera vaccines. In published cases of whole cell immunizations using C. difficile, however, the level of protection in experimental animals against CDI-induced death and non-lethal diarrhea was 40% or less. See, Torres et al., Infection and Immunity, 63(12):4619-27 (1995). The C. difficile combination vaccine compositions disclosed herein can be seen to provide a greater level of protective immune response than the use of either component alone.

The immunogenic compositions of the invention comprise an admixture of inactivated (i.e., killed) whole cells of C. difficile, or cell surface extracts (CSE), and a non-toxic C. difficile toxin polypeptide component, which may be a toxoid of C. difficile Toxin A, a toxoid of C. difficile Toxin B, an immunogenic fragment of Toxin A, an immunogenic fragment of Toxin B, or combinations of any or all of the foregoing polypeptides. The immunogenic compositions may employ whole cells and non-toxic polypeptides from one C. difficile strain or multiple C. difficile strains. In particular embodiments, the inactivated whole cell component, or cell surface extracts (CSE), are made from at least one hypervirulent strain of C. difficile such as a BI/NAP1/027 strain, and the non-toxic polypeptide component are made from at least one hypervirulent strain of C. difficile such as a BI/NAP1/027 strain. In particular embodiments, the non-toxic polypeptide component will be comprised of a Toxin B C-terminal fragment of TcdB2, especially TcdB2₁₆₅₁₋₂₃₆₆. In further embodiments, the vaccine composition will include an adjuvant, for example alum.

Inactivated C. difficile Cells

As used herein, “vaccine” is defined broadly to refer to any type of biological agent in an administrable form capable of stimulating an immune response in an animal inoculated with the vaccine that prevents or ameliorates the symptoms of CDI. Thus, reduction in the incidence or severity of characteristic symptoms of CDI, such as diarrhea, intestinal inflammation, necrosis of gastrointestinal tissues, or fluid accumulation in the gut in comparison to non-immunized subjects may be termed a vaccine for the purposes of this invention.

The terms “subject” and “patient” are used interchangeably herein and will be understood to refer to a warm-blooded animal, particularly a mammal. Non-limiting examples of animals within the scope and meaning of this term include guinea pigs, dogs, cats, rats, mice, horses, goats, cattle, sheep, zoo animals, monkeys, non-human primates, and humans.

For the purposes of the present invention, the term “infectious strain of C. difficile” refers to a strain that is capable of growing in a mammalian subject, e.g., in spite of any natural or man-made mutations that render the strain less virulent. For example, the whole cell component of the compositions described herein may utilize a wild type C. difficile, i.e., a strain found in nature, that is inactivated for use according to the invention. Alternatively, the inactivated whole cell component of the compositions described herein may advantageously utilize a non-toxinogenic strain of C. difficile, a sporulation deficient strain of C. difficile, a strain that is both non-toxinogenic and sporulation deficient, or another strain that is otherwise not pathogenic. Use of such non-pathogenic strains may be preferred, to ensure that no virulent fraction of a C. difficile culture is left after inactivation treatment that would have the potential of colonizing in a subject such as a human, leading to an unintended pathogenic infection.

The present invention provides novel vaccine compositions effective for controlling C. difficile infection in mammalian subjects. The present vaccine compositions are useful in providing immune resistance against the strain(s) of C. difficile used for preparation of the composition, as well as against strains which are different from those used in the preparation of the vaccine composition.

The particular strain or combination of strains of C. difficile used for preparation of the vaccine compositions described herein is not critical, however use of a hypervirulent strain of C. difficile such as BI/NAP1/027 as the source of the whole cell component and/or the Toxin B component is presumed to elicit an antibody response against antigenic sites associated with the most virulent C. difficile pathogens, and for that reason is to be preferred. Suitable C. difficile may be isolated from environmental or natural sources such patient isolates or from any of a wide range of culture deposits such as the American Type Culture Collection (ATCC, Manassas Va.). Particular C. difficile strains useful as sources for the composition components include, without limitation, VPI 10463 and other hypervirulent or non-hypervirulent strains of C. difficile, including but not limited to ribotypes 001, 003, 106, 012, 014, 027, 036, and 078. Hypervirulent strains of C. difficile suitable for use in the present invention include strains that have been classified as Group BI by restriction endonuclease analysis, as type NAP1 by pulsed-field gel electrophoresis, as ribotype 027 via PCR ribotyping, and/or as toxinotype III by toxin gene polymorphism typing. See, Merrigan et al., J. Bacteriol., 192(19):4904-4911 (2010). A suitable strain for culture of whole cells or as a source of Toxin A/B-encoding DNA for vector construction and recombinant production of a non-toxic Toxin A or Toxin B polypeptide fragment is the C. difficile type NAP1, toxinotype III strain deposited with ATCC (accession no. BAA-1870). The use of more than one C. difficile for preparing the inactivated cells component and the use of more than one Toxin A/B toxoid or Toxin A/B polypeptide fragment from one or more toxin proteins is specifically contemplated

Serum antibodies against C. difficile surface components are found in patients with CDI and are present in both symptomatic and asymptomatic carriers although the populations may differ. Such antibodies and differences in their populations are informative as to CSP preparations of the invention. For instance, based on a theory that an asymptomatic carrier is better able than a symptomatic carrier to block the most undesirable C. difficile-host interactions, in certain embodiments of the invention, a CSP composition of a vaccine is prepared or selected that binds to the repertoire of serum antibodies from an asymptomatic carrier or induces an antibody response like that of an asymptomatic carrier. In certain embodiments of the invention, in the preparation or selection of a CSP composition, CSP components that induce antibodies like those more abundant in a symptomatic carrier are avoided or selected against.

Propagation of the C. difficile bacterium for preparation of the compositions disclosed herein may be effected by culture under any conventional conditions employing media that support its growth. Although a variety of conventional solid and liquid media may be suitable for use herein, growth in liquid culture is particularly preferred for large scale production. One example of suitable liquid culture is the use of conventional tryptic soy broth supplemented with defibrinated sheep blood, cultured at 37° C. under an anaerobic gas mixture (e.g., 80% N₂/10% CO₂/10% H₂), however practitioners will be aware of many other media and culture conditions suitable for the uses described herein. After propagation of C. difficile in culture, whole cells are recovered by any suitable means, such as centrifugation or microfiltration.

Following their propagation and recovery, cells of C. difficile are subjected to chemical and/or physical treatment effective to inactivate (i.e., kill) the cells. An effective treatment for killing the cells is defined herein as that which kills 99% or more of the viable cells, preferably without disrupting the cells and while retaining the ability of the cells to elicit an antibody response in immunized mammals, including humans. The inactivation treatment should not substantially alter the specificity of the cell surface antigens on the killed cells relative to untreated cells. While treatments killing 100% of all viable cells would typically be preferred, the skilled practitioner will recognize 100% cell death may not always be readily obtainable. In the preferred embodiment, killed, intact C. difficile cells are prepared by treatment of viable cells with formalin. Alternatively, the cells may be killed by heat treatment, UV irradiation, or gamma irradiation. Any of a variety of other inactivation techniques that have been used for the preparation of killed cell vaccines or “bacterins” are also suitable for use herein, and these include but are not limited to treatment of cells with alcohols, particularly an aliphatic alcohol such as ethanol or isopropyl alcohol, phenol, tricresol, etc., treatment with formalin (aqueous formaldehyde solution, usu. 37%), formaldehyde, acetone, thimerosal (Merthiolate), β-lactam antibiotics, and the like. Heat treatment and other inactivation techniques preferably are carried out under conditions that do not cause surface protein denaturation. Suitable heat treatment, for example, will include, e.g., heating at 55°-65° C. for one hour, or heating at 80°−90° C. for 30 minutes (or less at the higher temperatures). Treatment times and conditions will of course vary with the particular method selected and may be readily determined by routine testing.

In one embodiment, C. difficile cells in their culture vessel are exposed to formalin for a sufficient period of time to kill >99% of the cells. Typically, formalin concentration in the culture media would range from about 1% to about 5% (v/v), preferably from about 1% to about 3% (v/v). Suitable exposure times for a particular formalin concentration to achieve 99%-100% killing may be readily determined from lethal killing curves of % killed vs. time of treatment.

Following fermentation and inactivation, the C. difficile cells are concentrated, for example, by filtration or centrifugation to obtain a high-density suspension of inactivated cells, and the cell pellet and fermentation culture fluid are separated. The separated cells may be retained for use as the first component of the vaccine composition. The filtrate, in the form of the cell-free culture fluid, is typically discarded, unless it is viewed as a source of intact Toxin B, which may be further treated to obtain a non-toxic Toxin B polypeptide for use as the second component of a combination vaccine composition according to the invention. While this is a possible procedure, it is not preferred to use treated full-length C. difficile Toxin B as a co-immunogen, because of the difficulty in separating Toxin B from culture and the risk of retaining intact Toxin B through the process, which may lead to unwanted toxicity from use of the composition.

Infection by C. difficile, aka, antibiotic associated diarrhea (AAD) often initiates following antibiotic treatment whereby germination of existing or ingested spores leads to colonization and expansion of vegetative cells. Ingested spores germinate in the colon, to establish a population of vegetative cells which produce cytotoxins and more spores. Infection develops when the normal intestinal microbiota is disturbed, for example by antibiotics, and C. difficile can colonize the gut. Two related toxins, TcdA and TcdB, act by glucosylating small GTPases including Rho, Rac and Cdc42 upon entry into host cells. The action of these toxins is responsible for the clinical manifestations of disease The C. difficile toxins are responsible for most of the disease symptoms, whereas the spores, which can remain latent in the gut, are both a persistence and transmission factor.

C. difficile possesses a highly deacetylated peptidoglycan cell wall containing unique secondary cell wall polymers. Bound to the cell wall is an essential S-layer, formed of SlpA and comprising 28 or more related proteins. In addition to the S-layer, many other cell surface proteins have been identified, including several with roles in host colonization. Kirk et al, 2017, Characteristics of the Clostridium difficile cell envelope and its importance in therapeutics, Microb Biotechnol. 10, 76-90; doi: 10.1111/1751-7915.12372)

In the spore, the genome is deposited in a central compartment delimited by a lipid bilayer with a layer of peptidoglycan (PG) apposed to its external leaflet. This layer of PG, known as the germ cell wall, serves as the wall of the outgrowing cell that forms when the spore completes germination. The germ cell wall is encased in a thick layer of a modified form of PG, the cortex, essential for the acquisition and maintenance of heat resistance. The cortex is wrapped by a multiprotein coat, which protects it from the action of PG-breaking enzymes produced by host organisms or predators. In C. difficile, the coat is further enclosed within a structure known as the exosporium. The coat and the exosporium, when present, mediate the immediate interactions of the spore with the environment, including the interaction with small molecules that trigger germination. (Pereira et al, 2013, The Spore Differentiation Pathway in the Enteric Pathogen Clostridium difficile, PLoS Genet. 9(10): e1003782, doi: 10.1371/journal.pgen.1003782)

Peptidoglycan (PG) is an essential component of the bacterial cell wall, functioning to maintain cell shape and integrity, and anchor cell wall proteins (CWP). PG structure is largely conserved, consisting of long glycan polymers cross-linked by short peptide chains. The polysaccharide backbone is composed of polymers of the β-1→4 linked disaccharide N-acetylglucosamine-N-acetylmuramic acid (GlcNAc-MurNAc).

Three anionic polysaccharides have been identified on the cell surface of C. difficile. The first (PS-I) consists of a branched penta-glycosylphosphate repeating unit, originally identified in a ribotype 027 strain and is only found in a minority of strains. The second (PS-II), a polymer of hexaglycosylphosphate repeat units (PS-II) and third (PS-III), a lipid bound glycosylphosphate polymer (PS-III), are more widely distributed. Glycans are T cell-independent antigens, although when conjugated to a carrier protein, these molecules can elicit a T-cell dependent memory response. (Kirk et al, 2017)

Cell Surface Extracts (CSE) Derived from C. difficile

In an embodiment of the invention, a cell surface extract (CSE) comprises an extract from whole C. difficile cells. Any C. difficile strain can be used to prepare a CSE, including without limitation any C. difficile strain identified herein. The CSEs comprise antigenic molecules, including but not limited to cell surface proteins and polysaccharides, that elicit protective immune responses. These partially purified extracts are advantageously amenable to characterization of the CSE components, for example by PAGE, ELISA, or chromatography and reproducible delivery of defined protein and polysaccharide components in a vaccine, and may be less reactogenic as compared to immunogenic compositions comprising inactivated whole cells. In certain embodiments a CSE is evaluated in a preclinical immunogenicity study, e.g. for potency and/or consistency.

Propagation of a C. difficile strain for preparation of the compositions of the invention may be affected by culture under any conventional conditions employing media that support its growth. Although a variety of conventional solid and liquid media may be suitable for use herein, growth in liquid culture is particularly preferred for large scale production. One example of suitable liquid culture is the use of conventional tryptic soy broth supplemented with defibrinated sheep blood, cultured at 37° C. under an anaerobic gas mixture (e.g., 80% N₂/10% CO₂/10% H₂), however practitioners will be aware of many other media and culture conditions suitable for the uses described herein. After propagation of C. difficile in culture, cells are recovered by any suitable means, such as centrifugation or microfiltration, and stored frozen at −80° C.

In an embodiment of the invention, to prepare a cell surface extract (CSE), frozen cell paste is thawed to ambient temperature then resuspended in 0.5% sodium deoxycholate at 1/50^{th} volume of the original culture volume, i.e. a 1000 mL culture would be resuspended in 20 mL of 0.5% sodium deoxycholate. The cell solution is then incubated at 60° C. for 16-24 hours with agitation, e.g. in a shaking incubator rotating at 225 rpm. After incubation, the cell solution is removed from the incubator and equilibrate to ambient temperature. Cells are subsequently separated from the solution by centrifugation (6000 rpm at 4° C. for 10 minutes) and the CSE containing supernatant is harvested. The CSE is then characterized for protein and polysaccharide content using a BCA protein assay and an anthrone assay, respectively. More particularly, in certain embodiments, component proteins and other cell surface molecules of the CSE are analyzed for amount and purity. Following characterization, the CSE is ready to be used to prepare an immunogenic composition.

### Cell Surface Molecules

C. difficile surface components play important roles not only in growth and survival but also in the interaction with the host and its immune system. All C. difficile strains express surface layer (S-layer) proteins (SLPs) on the outer cell surface, which are involved in adhesion to host intestinal cells, induction of cytokine production and the recognition of C. difficile by the immune system (Ryan et al, 2011, A role for TLR4 in Clostridium difficile infection and the recognition of surface layer proteins. PLoS Pathog 7:e1002076; Bianco et al, 2011, Immunomodulatory activities of surface-layer proteins obtained from epidemic and hypervirulent Clostridium difficile strains. J Med Microbiol 60:1162-7; Collins et al, Surface layer proteins isolated from Clostridium difficile induce clearance responses in macrophages. Microbes Infect 16:391-400). Examples of cell surface components include cell wall proteins (CWPs) and S-layer proteins (SLPs). Non-limiting examples of SLPs involved in colonization include adhesin Cwp66 (Waligora et al., 2001, Characterization of a cell surface protein of Clostridium difficile with adhesive properties. Infect. Immun. 69, 2144-2153. doi: 10.1128/IAI.69.4.2144-2153.2001) and protease Cwp84. The Cwp66 protein is one of a large family of gene products encoded by the C. difficile genome with significant homology to the highly expressed surface layer proteins (SLPs), encoded by the slpA gene (Calabi et al, 2001, Molecular characterization of the surface layer proteins from Clostridium difficile. Mol. Microbiol. 40:1187-1199; Karjalainen et al, 2001, Molecular and genomic analysis of genes encoding surface-anchored proteins from Clostridium difficile. Infect. Immun. 69:3442-3446). Cwp84 is surface exposed and conserved among strains, cleaves the SlpA precursor into the two mature SLPs (high molecular weight (HMW)-SLP and low-molecular weight (LMW)-SLP), and degrades collagen, fibronectin or vitronectin. (Janoir et al., 2007, Cwp84, a surface-associated protein of Clostridium difficile, is a cysteine protease with degrading activity on extracellular matrix proteins. J. Bacteriol. 189, 7174-7180. doi: 10.1128/JB.00578-07; Chapeton Montes et al., 2013, Influence of environmental conditions on the expression and the maturation process of the Clostridium difficile surface associated protease Cwp84. Anaerobe 19, 79-82. doi: 10.1016/j.anaerobe.2012.12.004). Another cell surface protein is the fibronectin-binding protein Fbp68 which is highly conserved between strains (Barketi-Klai et al, 2011, Role of fibronectin-binding protein A in Clostridium difficile intestinal colonization. J. Med. Microbiol. 60, 1155-1161. doi: 10.1099/jmm.0.029553-0).

Most, though not all, C. difficile strains are motile, involving cell surface flagellum and pillus proteins and structures which take part in intestinal colonization. The flagellin FliC and the cap protein FliD bind in vitro to murine mucus (Pėchinė et al, 2018, Targting Clostridium difficile Surface Components to Develop Immunotherapeutic Strategies Against Clostridium difficile Infection. Frontiers in Microbiology 9, 1-11). There are several pilin proteins and the N-terminal hydrophobic regions are relatively conserved while the C-termini are divergent (Maldarelli et al, 2014, Identification, immunogenicity, and cross-reactivity of type IV pilin and pilin-like proteins from Clostridium difficile, Pathog. Dis. 71, 302-314. doi: 10.1111/2049-632X.12137).

Cell surface proteins and encoding genes include, without limitation, the following:

TABLE 1
C. difficile cell surface extract proteins*

| Cell wall | | | Mol/mass | GenBank |
|---|---|---|---|---|
| gene name | Gene name | Gene number | (kDa) | Accession No. |
| slpA | Dif205 | CD2793 | 76.1 | RKM67916.1 |
| cwp2 | | CD2791 | 66.5 | CAJ69679.1 |
| cwp66 | Dif204 | CD2789 | 66.8 | CAJ69677.1 |
| cwp84 | | CD2787 | 87.3 | CAJ69675.1 |
| cwp5 | Dif146 | CD2786 | 57.2 | CAJ69674.1 |
| cwp6 | Dif145 | CD2784 | 73.0 | CAJ69672.1 |
| cwp7 | Dif144 | CD2782 | 39.3 | CAJ69670.1 |
| cwp8 | | CD2799 | 68.5 | CAJ69687.1 |
| cwp9 | | CD2798 | 52.5 | CAJ69686.2 |
| cwp10 | Dif207 | CD2796 | 67.0 | CAJ69684.2 |
| cwp11 | Dif149 | CD2795 | 58.7 | CAJ69683.1 |
| cwp12 | | CD2794 | 57.5 | CAJ69682.1 |
| cwp13 | Dif196 | CD1751 | 87.2 | CAJ68619.1 |
| cwp14 | | CD2735 | 51.4 | CAJ69622.1 |
| cwpV | Dif189A; Dif189B | CD0514 | 166.7 | CAJ67348.1 |
| cwp16 | Dif192 | CD1035 | 74.7 | CAJ67876.2 |
| cwp17 | | CD1036 | 74.2 | CAJ67877.2 |
| cwp18 | Dif53 | CD1047 | 37.4 | CAJ67888.1 |
| cwp19 | | CD2767 | 77.8 | CAJ69655.1 |
| cwp20 | Dif75; Dif75A; | CD1469 | 111.1 | CAJ68334.1 |
| | Dif75B | | | |
| cwp21 | Dif211 | CD3192 | 61.1 | CAJ70089.1 |
| cwp22 | | CD2713 | 72.0 | CAJ69599.2 |
| cwp23 | | CD1803 | 63.8 | CAJ68673.1 |
| cwp24 | Dif106 | CD2193 | 51.3 | CEJ98797.1 |
| cwp25 | Dif44 | CD0844 | 33.8 | CAJ67678.1 |
| cwp26 | | CD1233 | 52.0 | CAJ68087.1 |
| cwp27 | | CD0440 | 41.0 | CAJ67265.1 |
| cwp28 | | CD1987 | 51.7 | CAJ68862.1 |
| cwp29 | Dif201 | CD2518 | 50.0 | CAJ69405.1 |

*Accession numbers of exemplary proteins of C. difficile strains.

Table 1 provides exemplary protein sequences of C. difficile strain 630. The invention includes all strains of C. difficile and proteins, extracts, and CSEs therefrom.
The domain architectures of the encoded proteins have been determined along with determined or putative functions. Many of the Cwp proteins are paralogs, apparently derived from the same ancestral protein by duplication and evolved to a new role or function. (See e.g., Fagan et al., 2011, A proposed nomenclature for cell wall proteins of Clostridium difficile, J Med. Microbiol. 60:1225-1228).

Cell surface extracts can also comprise:

GroEL: This Hsp60 protein is partially membrane bound. GroEL-specific antibodies as well as the purified GroEL protein partially inhibit C. difficile cell attachment.

CD3246: This protein comprises a putative cell-wall anchored adhesin.

CD2381: This protein comprises a putative cell-wall anchored adhesion.

CD0873: This comprises a cell-surface lipoprotein with a role in adhesion.

CbpA: This protein is also known as “CD3145” and comprises a collagen binding protein.

Dif44: This protein is also known as “CD0844” and comprises cell surface protein cwp25 from C. difficile.

Dif51: This protein is also known as “CD0999” and comprises an ABC transporter substrate binding protein lipoprotein from C. difficile.

Dif1301 This protein is also known as “CD2645” and comprises a putative extracellular solute binding protein from C. difficile.

Dif192: This protein is also known as “CD1035” and comprises cell surface protein cwp16 (putative Nacetylmuramoyl-L-alanine amidase) from C. difficile.

Dif208, Dif208A: This protein is also known as “CD2831” and comprises collagen-binding protein from C. difficile.

Dif232: This protein is also known as “CD1031” and comprises a cell wall anchored protein from C. difficile.

CDT: This protein is a toxin frequently produced by hypervirulent strains of C. difficile such as BI/NAP1/027.

In an embodiment of the invention, individual components of CSEs are purified or recombinantly expressed. In an embodiment of the invention, one or more purified component, for example, 1, 2, 3, 4, 5, 6, 7, 8, 9, 10, or more components are added to a CSE to boost the level of an antigen of interest. In an embodiment of the invention, one or more purified component, for example 1, 2, 3, 4, 5, 6, 7, 8, 9, 10, or more components are combined to produce a CSE.

C. difficile surface components include polysaccharides and lipoteichoic acid. PS-I is a polymer of branched pentasaccharide phosphate-repeats composed of rhamnose and glucose but is expressed in low levels and consequently difficult to obtain. Immunogenic PS-I can be synthesized (Martin et al, 2013, Immunological evaluation of a synthetic Clostridium difficile oligosaccharide conjugate vaccine candidate and identification of a minimal epitope. J. Am. Chem. Soc. 135, 9713-9722. doi: 10.1021/ja401410y). PS-II, which is conserved across most C. difficile strains, is a polymer of hexasaccharide phosphate-repeats composed of glucose, mannose, N-aceylgalactosamine (Ganeshapillai et al, 2008, Clostridium difficile cell-surface polysaccharides composed of pentaglycosyl and hexaglycosyl phosphate repeating units. Carbohydr. Res. 343, 703-710. doi: 10.1016/j.carres.2008.01.002). In many cases, to be immunogenic, polysaccharides need to be coupled to carrier proteins. For example, both native PS-II and a synthetic PS-II hapten conjugated to diphtheria toxoid are immunogenic (Adamo et al, 2012, Phosphorylation of the synthetic hexasaccharide repeating unit is essential for the induction of antibodies to Clostridium difficile PSII cell wall polysaccharide. ACS Chem. Biol. 7, 1420-1428. doi: 10.1021/cb300221f).

The protein and polysaccharide components of CSEs in therapeutic and prophylactic compositions of the invention can be present in different ratios. In embodiments of the invention, suitable ratios of protein to polysaccharide can include, without limitation, about 1:100, 1:50, 1:20, 1:10, 1:5, 1:4, 1:3, 1:2, 1:1, 2:1, 3:1, 4:1, 5:1, 19:1, 20:1, 50:1, or 100:1 by weight.

Non-Toxic C. difficile Toxoids, Toxin a Fragments, and Toxin B Fragments

The second component of the immunogenic composition according to the invention is a non-toxic polypeptide comprising a toxoid of C. difficile Toxin A or Toxin B, an immunogenic portion of C. difficile Toxin A or Toxin B, or combinations thereof. The TcdA (308 kD Toxin A) and TcdB (269 kD Toxin B) proteins belong to a large clostridial cytotoxin (LCT) family and share 49% amino acid identity (Just, I. et al. 2004 Rev Physiol Biochem Pharmacol 152:23-47). The tcdA and tcdB genes and three accessory genes are located on the bacterial chromosome, forming a 19.6-kb pathogenicity locus (PaLoc) (142). TcdA and TcdB are structurally similar to each other (von Eichel-Streiber, C. et al. 1996 Trends Microbiol 4:375-382), comprising at least three functional domains. The C-terminal region of both TcdA and TcdB is responsible for toxin binding to the surface of epithelial cells. The C-terminus contains a receptor binding domain (RBD) and has a β-solenoid structure (Ho, J. G. et al. 2005 Proc Natl Acad Sci USA 102: 18373-1837). The middle portion of the toxin primary structure is potentially involved in translocation of the toxin into target cells, and the N-terminus is a catalytic domain having glucosyltransferase activity (Hofmann, F. et al. 1997 J Biol Chem 272: 11074-11078). The crystal structure of RBD of TcdA revealed a solenoid-like structure. The boundary of the RBD in both toxins is near amino acid 1850. Interaction between the C-terminus and the host cell receptors is believed to initiate receptor-mediated endocytosis (Florin, I. et al. 1983 Biochim Biophys Acta 763:383-392; Karlsson, K. A. 1995 Curr Opin Struct Biol 5:622-635; Tucker, K. D. et al. 1991 Infect Immun 59:73-78).

Binding of TcdA/B to epithelial cells induces receptor-mediated endocytosis and entry into the cytoplasm. Once internalized, a decrease in endosomal pH is thought to induce a conformational change which results in exposure of the hydrophobic translocation domain and insertion of the enzymatic N-terminus (comprising a glycosyl-transferase domain (“GT”) and a cysteine protease domain (“CP”)), allowing entry into the endosome via pore formation. Upon cleavage, the GT domain is thought to be capable of transferring glucose residues from UDP-glucose to Rho-GTPases, thus inactivating cell signaling. Among other effects, inhibition of Rho-GTPases causes dysregulation of the actin cytoskeleton and tight junctions and leads to increased membrane permeability and loss of barrier function, resulting in diarrhea, inflammation, and an influx of neutrophils and other members of the innate immune response.

Where a full-length Toxin A or Toxin B protein is to be included in the immunogenic composition, it should be rendered a toxoid, that is, inactivated (made non-toxic) but retaining the ability to elicit a protective immune response producing antibodies that recognize (bind to) the toxin from which the toxoid is made. Any method for inactivating the toxin may be used, but typically the full-length toxin is treated with formaldehyde or heat until the toxin is rendered a toxoid.

In other vaccine compositions, non-toxic fragments of Toxin A and/or Toxin B are employed, for example, fragments lacking the glucosyltransferase activity encoded at the N-terminal of the toxins. Where a non-toxic fragment of Toxin A or Toxin B is to be used, a sufficient portion of the Toxin A or Toxin B protein should be used that is capable to elicit a protective immune response producing antibodies recognizing a native Toxin A or Toxin B.

TcdA (308 kDa, Acc.Nr. P16154) and TcdB (270 kDa, Acc.Nr. P18177) have 2,710 and 2,366 amino acids, respectively, and are 48% identical in their amino acid sequence. The toxins consist of at least four major functional domains according to the Activity-Cutting-Delivery-Binding (ABCD) model (Jank et al, 2008, Structure and mode of action of clostridial glucosylating toxins: the ABCD model. Trends Microbiol. 16:222-29). The N-terminal, biologically active domain A harbors glucosyltransferase activity that modifies Rho proteins of an infected cell. The C-terminal part of the toxin is involved in receptor binding. The C (cutting) domain follows domain A and possesses protease function. The D domain is primarily involved in delivery of the toxin (or N-terminal part of the toxin) into the cytosol of target cells.

The binding (B) domains of TcdA and TcdB are usually defined to cover residues 1,832-2,710 and 1,834-2,366, respectively, and are characterized by repetitive sequences called combined repetitive peptides (CROPs), comprising a solenoid fold with 7 long repeats of 30 residues and 31 short repeats of 15-21 residues in TcdA and 7 long repeats of 30 residues and 21 short repeats of 20-23 residues in TcdB (Ho et al, 2005, Crystal structure of receptor-binding C-terminal repeats from Clostridium difficile toxin A. PNAS 102:18373-78; Murase et al, 2014, Structural basis for antibody recognition in the receptor-binding domains of toxins A and B from Clostridium difficile. J. Biol. Chem. 289:2331-43.

A variety of modified or truncated Toxin A and B polypeptides have been described which have been shown to be both non-toxic and immunogenic. See, e.g., WO 2011/068953, WO 2013/040254, WO 2014/176276, Aktories et al., 2017, Clostridium difficile Toxin Biology, Annu. Rev. Microbiol. 71:281-307.

While any immunogenic and non-toxic Toxin A or Toxin B polypeptide fragment may be used in compositions according to this invention, particular mention is made of polypeptides derived from the C-terminal portion of Toxin A and Toxin B, which contains the combined repetitive oligopeptides (CROPs) region, which comprises cell surface receptor binding sites in both toxins. In a particular embodiment, an immunogenic fragment of Toxin A or Toxin B elicits antibodies that bind to the cell surface receptor binding site of Toxin A or Toxin B and block binding to the receptor The CROPs region has been shown to be highly immunogenic, and therefore the C-terminal region of C. difficile Toxin A or Toxin B containing combined repetitive oligopeptides (“CROPs regions”) is of particular interest for the practice of the present invention. The polypeptide comprised of amino acids 1651 to 2366 of the full-length Toxin B of a C. difficile strain is a preferred segment of the Toxin B protein to include when selecting a non-toxic C. difficile toxin polypeptide fragment. The polypeptide comprised of amino acids 1649 to 2710 of the full-length Toxin A of a C. difficile strain is a preferred segment of the Toxin A protein to include when selecting a non-toxic C. difficile toxin polypeptide fragment. Another useful segment of C. difficile Toxin B is the CROP region from amino acid 1834 to 2366. Another useful segment of C. difficile Toxin A is the CROP region from amino acid 1832 to 2710.

In an embodiment of the invention, a non-toxic C. difficile toxin polypeptide fragment comprises a portion of Toxin B which is an epitope for an anti-toxin B antibody. Fragments comprising such an epitope include, without limitation, SPNIYTDEINITPVYETN (SEQ ID NO:7), YPEVIVLDANYINEKI (SEQ ID NO:8), TVGDDKYYFNPINGG (SEQ ID NO:9), ASIGETIIDDKNYYFNQS (SEQ ID NO:10), EDGFKYFAPANTLDEN (SEQ ID NO:11) PANTLDENLEGE (SEQ ID NO:12), AIDFTGKLIIDE (SEQ ID NO:13), NIYYFDDNYRGAVE (SEQ ID NO:14), HYFSPETGKAFK (SEQ ID NO:15), IGDYKYFNSDGVM (SEQ ID NO:16), HFYFAENGEMQIGVFNTEDGFK (SEQ ID NO:17), INDGQYYFNDDGIMQV (SEQ ID NO:18), YKYFAPANTVNDNIYG (SEQ ID NO:19), ESDKYYFNPETKKA (SEQ ID NO:20), NNNYYFNENGEMQFGYINI (SEQ ID NO:21), and QNTLDENFEGESINYT (SEQ ID NO:22) from the 10463 strain and SPNIYTDEINITPIYEAN (SEQ ID NO:23), YPEVIVLDTNYISEKI (SEQ ID NO:24), TIGDDKYYFNPDNGG (SEQ ID NO:25), ASVGETIIDGKNYYFSQN (SEQ ID NO:26), EDGFKYFAPADTLDEN (SEQ ID NO:27), PADTLDENLEGE (SEQ ID NO:28), AIDFTGKLTIDE (SEQ ID NO:29), NVYYFGDNYRAAIE (SEQ ID NO:30), YYFSTDTGRAFK (SEQ ID NO:31), IGDDKFYFNSDGIM (SEQ ID NO:32), YFYFAENGEMQIGVFNTADGFK (SEQ ID NO:33), INDGKYYFNDSGIMQI (SEQ ID NO:34), YKYFAPANTVNDNIY (SEQ ID NO:35), ESDKYYFDPETKK A (SEQ ID NO:36), D NHYYFNEDGIMQYGYLNI (SEQ ID NO:37), and QNTLDENFEGESINYT (SEQ ID NO:38) from the BI/NAP1/027 strain.

In another embodiment of the invention, a non-toxic C. difficile toxin polypeptide fragment comprises amino acids 2152-2341 of toxin B from the 10463 strain: DDNGIVQIGVFDTSDGYKYFAPANTVNDNIYGQAVEYSGLVRVGEDVYYFGETYTIETG WIYDMENESDKYYFNPETKKACKGINLIDDIKYYFDEKGIMRTGLISFENNNYYFNENG EMQFGYINIEDKMFYFGEDGVMQIGVFNTPDGFKYFAHQNTLDENFEGESINYTGWLDL DEKRYYFTDEYIA (SEQ ID NO:39). In still another embodiment, a non-toxic C. difficile toxin polypeptide fragment comprises amino acids 2152-2341 of toxin B from the BI/NAP1/027 strain:

(SEQ ID NO: 40)
DENGLVQIGVFDTSDGYKYFAPANTVNDNIYGQAVEYSGLVRVGEDVYYF
GETYTIETGWIYDMENESDKYYFDPETKKAYKGINVIDDIKYYFDENGIM
RTGLITFEDNHYYFNEDGIMQYGYLNIEDKTFYFSEDGIMQIGVFNTPDG
FKYFAHQNTLDENFEGESINYTGWLDLDEKRYYFTDEYIA.
(See WO 2013/040254).

In another embodiment, a non-toxic C. difficile toxin polypeptide comprises a Toxin B epitope that binds to bezlotoxumab. The two Fab regions of bezlotoxumab bind to TcdB at two distinct sites of the C. difficile strain VPI 10463 TcdB CROP domain spanning two CROP units. One site (“E1”) consists of a discontinuous epitope within amino acids 1806-1961 and the second site (“E2”) consists of a discontinuous epitope within amino acids 2007-2093. Notably, of the 18 bezlotoxumab-interacting residues in common between E1 and E2, only 10 are identical, although 6 of the 8 amino acids substitutions are conservative. (Orthe et al, 2015, Mechanism of Action and Epitopes of Clostridium difficile Toxin B-neutralizing Antibody Bezlotosumab Revealed by X-ray Crystallography, J. Biol. Chem., 289:18008-18021.

In another embodiment, a non-toxic C. difficile toxin polypeptide comprises a Toxin A epitope that binds to actoxumab. There are two distinct actoxumab binding sites within the CROP domain of TcdA centered on identical amino acid sequences at residues 2162-2189 and 2410-2437. (Hernandez et al, 2017, Epitopes and Mechanism of Action of the Clostridium difficile Toxin A-Neutralizing Antibody Actoxumab, J. Mol. Biol. 429:1030-1044.)

In alternative embodiments, a non-toxic, immunogenic polypeptide fragment of a C. difficile Toxin A or Toxin B comprises at least one mutation, for example, a deletion, substitution, insertion, or truncation that renders the protein non-toxic. In certain embodiments, an immunogenic polypeptide or polypeptide fragment of a C. difficile Toxin A comprises or Toxin B comprises at least one mutation in the glucosyl-transferase domain (amino acids 1-541 of SEQ ID NO:4 for C. difficile strain VPI 10463 Toxin A; amino acids 1-541 of SEQ ID NO:5 for C. difficile strain BI/NAP1/027 Toxin A; amino acids 1-543 of SEQ ID NO:1 for C. difficile strain VPI 10463 Toxin B; amino acids 1-543 of SEQ ID NO:2 for C. difficile strain BI/NAP1/027 Toxin B).

Detoxification of Toxin B may be achieved by mutating the amino acid sequence or the encoding nucleic acid sequence of the glucosyl-transferase domain of the wild-type Toxin B antigen using any appropriate method known in the art e.g. site-directed mutagenesis. In embodiments of the invention, the mutated Toxin B antigen comprises one or more amino acid substitutions (i.e. 1, 2, 3, 4, 5, 6, 7, 8, 9, 10, 11, 12, 13, 14, 15, 16, 17, 18, 19, 20, 25, 30, or more mutations), relative to the wild-type Toxin B antigen sequence of SEQ ID NO:1 or 2. For example, the mutated antigen may comprise substitutions at 1, 2, 3, 4 or 5 positions corresponding to amino acids 270, 273, 284, 286 and/or 288 of the Toxin B antigen sequence of SEQ ID NO:1 or 2.

Detoxification of Toxin A may be achieved by mutating the amino acid sequence or the encoding nucleic acid sequence of the glucosyl-transferase domain of the wild-type Toxin A antigen using any appropriate method known in the art e.g. site-directed mutagenesis. In embodiments of the invention, the mutated Toxin A antigen comprises one or more amino acid substitutions (i.e. 1, 2, 3, 4, 5, 6, 7, 8, 9, 10, 11, 12, 13, 14, 15, 16, 17, 18, 19, 20, 25, 30, or more mutations), relative to the wild-type Toxin A antigen sequence of SEQ ID NO:4 or 5. For example, the mutated antigen may comprise substitutions at 1, 2 or 3 positions corresponding to amino acids 283, 285 and 287 of the Toxin A antigen sequence of SEQ ID NO:4 or 5.

In certain embodiments, an immunogenic polypeptide or polypeptide fragment of a C. difficile Toxin A comprises or Toxin B comprises at least one mutation in the cutting or cysteine protease enzymatic domain (amino acids 542-769 of SEQ ID NO:4 for C. difficile strain VPI 10463 Toxin A; amino acids 542-769 of SEQ ID NO:5 for C. difficile strain BI/NAP1/027 Toxin A; amino acids 544-767 of SEQ ID NO:1 for C. difficile strain VPI 10463 Toxin B; amino acids 544-767 of SEQ ID NO:2 for C. difficile strain BI/NAP1/027 Toxin B) that renders the protease inactive.

In certain embodiments, an immunogenic polypeptide or polypeptide fragment of a C. difficile Toxin A or Toxin B comprises at least one mutation in the translocation domain (amino acids 770-1808 of SEQ ID NO:4 for C. difficile strain VPI 10463 Toxin A; amino acids 770-1808 of SEQ ID NO:5 for C. difficile strain BI/NAP1/027 Toxin A; amino acids 768-1833 of SEQ ID NO:1 for C. difficile strain VPI 10463 Toxin B; amino acids 768-1833 of SEQ ID NO:2 for C. difficile strain BI/NAP1/027 Toxin B) that renders the protease inactive.

In alternative embodiments, a non-toxic, immunogenic polypeptide fragment of a C. difficile Toxin A comprises a minimum length of 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, or 2650 amino acids of Toxin A. In alternative embodiments, a non-toxic, immunogenic polypeptide fragment of a C. difficile Toxin B comprises a minimum length of 10, 20, 30, 40, 50, 60, 70, 80, 90, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, or 2350 amino acids of Toxin B, or Toxin A from amino acid 1649-2710, or Toxin B from amino acid 1651-2366) or from the CROP region of Toxin A (aa 1832-2710) or Toxin B (aa 1834-2366). The Toxin A or Toxin B sequences can be from strain 10463, hypervirulent strain BI/NAP1/027, or any other C. difficile strain.

In alternative embodiments or the invention, a non-toxic, immunogenic polypeptide of a C. difficile Toxin A or Toxin B comprises two or more Toxin A or Toxin B fragments which are the same or different, contiguous or non-contiguous, and linked in any order. For example, where a hybrid polypeptide comprises two TcdA antigens and one TcdB antigen, they may be in the order A-A-B, A-B-A, B-A-A from N-terminus to C-terminus, or where a hybrid polypeptide comprises two TcdB antigens and one TcdA antigen, they may be in the order B-B-A, B-A-B, A-B-B from N-terminus to C-terminus. In general, TcdA and TcdB antigens may alternate e.g. A-B-A or B-A-B.

In alternative embodiments of the invention the toxin proteins or fragments have at least 50%, at least 55%, at least 60%, at least 65%, at least 70%, at least 75%, at least 80%, at least 85%, at least 90%, at least 95%, at least 96%, at least 97%, at least 98%, or at least 99% identity to the Toxin A or Toxin B sequences or fragments set forth herein.

The terms “percent similarity,” “percent identity,” or “percent homology,” when referring to a particular sequence are used as set forth in the University of Wisconsin GCG software program.

In preparing the second component of the immunogenic composition according to the invention, inactivated full-length Toxin A or Toxin B or a non-toxic Toxin A or Toxin B polypeptide fragment from one or more strains of C. difficile may be used. Additionally, the second component may be comprised of one or more toxoids, one or more toxin polypeptide fragments, or combinations of toxoid and polypeptide fragment materials may be used. Thus, by way of non-limiting example, an immunogenic composition according to the invention may be comprised of component (a) inactivated C. difficile cells of one or more strains of C. difficile, e.g., a mixture of inactivated C. difficile VPI 10463 cells and inactivated C. difficile BI/NAP1/027 cells, plus component (b) a toxoid of Toxin A from VPI 10463, a toxoid of Toxin B from VPI 10463, a toxoid of Toxin A from BI/NAP1/027, a toxoid of Toxin B from BI/NAP1/027, a polypeptide comprising a C-terminal fragment of Toxin A or B (e.g., a CROPs region of Toxin A or B) from C. difficile strain VPI 10463, a polypeptide comprising a C-terminal fragment of Toxin A or B (e.g., a CROPs region of Toxin A or B) from a C. difficile BI/NAP1/027 strain, or combinations of more than one of such toxoids and polypeptide fragments. A preferred polypeptide fragment containing CROPs regions is a polypeptide comprising the C-terminal 716 amino acids of C. difficile Toxin B, in particular amino acids 1651-2366 of Toxin B from a hypervirulent strain (Toxin B_{HV}), such as Toxin B from a C. difficile BI/NAP1/027 strain (TcdB2).

It is preferred that any non-toxic polypeptide fragment used in the second component of the claimed immunogenic compositions will be a recombinantly produced polypeptide, which will simplify purification and avoid retained toxicity issues. A recombinant structural gene encoding a Toxin A or B polypeptide fragment may be readily synthesized to order for insertion into a variety of bacterial expression systems, and the polypeptide product may be readily isolated by conventional methods known to practitioners in the field.

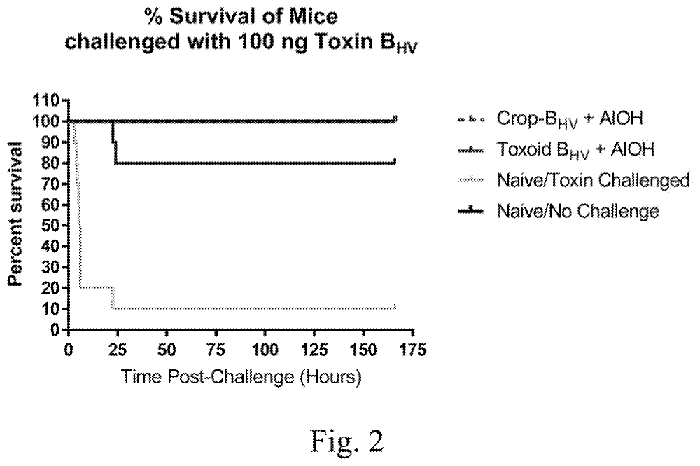

In an experiment evaluating protection against Toxin B_{HV} challenge, CROPB_{HV} immunized mice were completely protected against toxin challenge (10 of 10 mice) whereas 8 of 10 Toxoid B_{HV} immunized mice were protected against toxin challenge (Examples; Table 3; FIG. 2) demonstrating that CROPB_{HV} is a highly effective immunogen comparable to or better than full-length, inactivated toxin B_{HV}.

### Preparation of Immunogenic Compositions

An immunogenic composition according to the invention may be prepared by simple admixture of component (a), e.g., a suspension (e.g., resuspended pellet) of inactivated C. difficile whole cells or CSP preparation and component (b), e.g., a solution of at least one Toxin A or B toxoid and/or at least one non-toxic C. difficile Toxin A or Toxin B polypeptide. Appropriate ratios of the components (a) and (b) may be determined by those skilled in the art. In certain embodiments, sufficient inactivated whole cell (WC) or cell surface extract (CSE) component (a) to provide 10⁷-10¹¹ cells or 10-100 μg of CSE, based on polysaccharide amount, should be used; and sufficient toxoid/toxin polypeptide fragment component (b) to provide 4-500 μg of toxoid or non-toxic polypeptide fragment should be used. Other amounts may be used if multiple immunizations are contemplated. The amounts of the components may be independently adjusted at the option of the practitioner to affect the anti-C. difficile antibody or the anti-Toxin A/B antibody titer resulting from immunizing a subject with the combination vaccine composition.

The immunogenic composition containing at least components (a) and (b) described above may be prepared for administration by formulation in an immunologically effective amount or dosage. The formulation may further include pharmaceutically acceptable carriers and adjuvants known in the art.

An immunologically effective amount or dosage is defined herein as being that amount which will induce complete or partial immunity (elicit a protective immune response) in an immunized mammalian subject. Immunity is considered as having been induced in a population of treated subjects when the level of protection for the population (evidenced by a decrease in the number of CDI symptoms or a decrease in the severity of CDI symptoms) is significantly higher than that of an unvaccinated control group (measured at a confidence level of at least 80%, preferably measured at a confidence level of 95%). The appropriate effective dosage can be readily determined by practitioners in this field by routine experimentation.

A suitable therapeutic dose of C. difficile cells comprises from about 10⁷ to about 10¹¹ cells, or from about 10⁷ to about 10⁸ cells, or from about 10⁸ to about 10⁹ cells, or from about 10⁹ to about 10¹⁰ cells, or from about 10¹⁰ to about 10¹¹ cells. A suitable therapeutic dose of C. difficile cell surface extract (CSE) comprises from about 0.001 μg/kg to about 100 mg/kg, or from about 0.01 μg/kg to about 1 mg/kg, or from about 0.1 μg/kg to about 10 μg/kg, or from about 1 to about 5 μg/kg, or from about 5 to about 10 μg/kg, from about 10 to about 50 μg/kg, from about 50 to about 100 mg/kg, from about 100 to about 500 μg/kg, from about 500 μg/kg to about 1 mg/kg, from about 1 to about 5 mg/kg, from about 5 to about 10 mg/kg, from about 10 to about 50 mg/kg, or from about 50 to about 100 mg/kg. In therapies of the invention a suitable dose of CROPB_{HV} or TcdA or TcdB2, or Toxin A or B toxoid comprises from about 5 μg/kg to about 100 mg/kg, or from about 5 to about 10 μg/kg, from about 10 to about 50 μg/kg, from about 50 to about 100 mg/kg, from about 100 to about 500 μg/kg, from about 500 μg/kg to about 1 mg/kg, from about 1 to about 5 mg/kg, from about 5 to about 10 mg/kg, from about 10 to about 50 mg/kg, or from about 50 to about 100 mg/kg.

Typically, the immunogenic composition will contain at least 1×10⁸ cells of C. difficile or 25 μg of C. difficile CSE, and at least 5 μg of Toxin A and/or B toxoid or non-toxic Toxin A and/or B polypeptide.

The doses may be more or less depending on age, health status, history of prior infection, and immune status of the subject as would be known by one of skill in the art of immunization. Doses may be divided or unitary per day and may be administered once or repeated at appropriate intervals.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Suitable pharmaceutically acceptable carriers for suspension of the killed cells include but are not limited to water, physiological saline, mineral oil, vegetable oils, aqueous sodium carboxymethyl cellulose, or aqueous polyvinylpyrrolidone. The two-component vaccine formulations may also contain optional adjuvants, antibacterial agents or other pharmaceutically active agents as are conventional in the art. Without being limited thereto, suitable adjuvants include but are not limited to mineral oil, vegetable oils, alum, Freund's incomplete adjuvant, Freund's complete adjuvant, QS-21, salts, e.g., AlK(SO₄)₂, AlNa(SO₄)₂, AlNH₄(SO₄)₂, silica, kaolin, muramyl dipeptide, carbon polynucleotides, e.g., poly IC and poly AU, and QuilA and Alhydrogel, microparticles or beads of biocompatible matrix materials (e.g., agar, polyacrylate). Practitioners will recognize that other carriers or adjuvants may be used as well.

As set forth above, the invention provides compositions, vaccines and components thereof comprising C. difficile cells, C. difficile extracts, C. difficile cell surface preparations, toxoids or fragments of C. difficile toxin A and/or toxin B, and combinations thereof.

The immunogenic compositions of the invention may be administered to the subject mammal by any convenient route which enables the killed cells, extracts or cell surface preparations and toxoid(s)/toxin polypeptide fragment(s) to elicit an immune response, such as but not limited to intraperitoneal (i.p.), intramuscular (i.m.), or subcutaneous (s.c.) injection, oral administration, or mucosal (e.g., intranasal (i.n.), intragastric (i.g.), intravaginal, rectal (r)) administration.

The immunogenic compositions can be introduced into the gastrointestinal tract or the respiratory tract, e.g., by inhalation of a solution or powder containing the conjugates. In some embodiments, the compositions can be administered via absorption via a skin patch. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained.

The components may be administered together or separately, by the same route or by different routes. The components may be administered concomitantly, or they may be administered sequentially, i.e., one before the other.

In an embodiment, intramuscular injection is preferred for primary immunization. Secondary or booster immunization can be by the same or different route. Thus, in an embodiment of the invention, intramuscular injection is preferred for primary immunization and secondary or booster immunization. In another embodiment, intramuscular injection is preferred for primary immunization, while oral immunization is used for secondary or booster immunization, for example as preferred or convenient. The compositions may be administered in a single dose or in a plurality of doses. Where the composition is prescribed for administration in multiple doses, the timing and amounts of doses may be readily determined by the skilled practitioner.

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and from animal studies are used in formulating a range of dosage for human use.

Dosage can be by a single dose schedule or a multiple dose schedule. The components can be administered together in the same composition or in different compositions. Multiple doses of each component may be used in a primary immunization schedule and/or in a booster immunization schedule. The multiple doses of the components can be administered in combination or separately, for example at different times or by different routes of administration. In a multiple dose schedule the various doses may be given by the same or different routes, e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks apart, etc.).

Vaccines produced by the invention may be administered prophylactically to patients at substantially the same time as (e.g. during the same medical consultation or visit to a healthcare professional or vaccination center, or prior to a scheduled hospitalization) other vaccines e.g. at substantially the same time as a pneumonia vaccine, measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

In certain embodiments, a vaccine of the invention is administered therapeutically. In an embodiment, the vaccine is administered to a patient with an active C. difficile infection to both treat the active infection and/or prevent recurrence of a CDI. Without being bound by theory, the vaccines are believed to improve the immune status of the infected host by stimulating specific immune responses against C. difficile cell surface molecules and toxins that block or inhibit interactions of the C. difficile cells with cells of the host. Thus, therapeutic doses can be effectively administered even after initiation of a CDI. In certain embodiments, therapeutic administration of a vaccine is used to reduce or prevent recurrent CDI. Without being bound by theory, it is believed vaccines of the invention elicit immune responses against particular C. difficile components that mediate host cell interactions, and thus block reinfection or recurrence.

The compositions of the invention may elicit both a cell mediated immune response as well as a humoral immune response. This immune response will preferably induce long lasting (e.g. neutralising) antibodies and a cell mediated immunity that can quickly respond upon exposure to C. difficile. Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognize or interact with antigens displayed on MHC Class I molecules.

CD4 T cells can express a CD4 co-receptor and are commonly referred to as T helper cells. CD4 T cells are able to recognize antigenic polypeptides bound to MHC class II molecules. Upon interaction with an MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: T_{H}1 phenotype and T_{H}2 phenotypes which differ in their cytokine and effector function.

Activated T_{H}1 cells enhance cellular immunity (including an increase in antigen-specific CTL production) and are therefore of particular value in responding to intracellular infections. Activated T_{H}1 cells may secrete one or more of IL-2, IFN-γ, and TNF-β. A T_{H}1 immune response may result in local inflammatory reactions by activating macrophages, NK (natural killer) cells, and CD8 cytotoxic T cells (CTLs). A T_{H}1 immune response may also act to expand the immune response by stimulating growth of B and T cells with IL-12. T_{H}1 stimulated B cells may secrete IgG2a.

Activated T_{H}2 cells enhance antibody production and are therefore of value in responding to extracellular infections. Activated T_{H}2 cells may secrete one or more of IL-4, IL-5, IL-6, and IL-10. A T_{H}2 immune response may result in the production of IgG1, IgE, IgA and memory B cells for future protection.

An enhanced immune response may include one or more of an enhanced T_{H}1 immune response and a T_{H}2 immune response. A T_{H}1 immune response may include one or more of an increase in CTLs, an increase in one or more of the cytokines associated with a T_{H}1 immune response (such as IL-2, IFN-γ, and TNF-β), an increase in activated macrophages, an increase in NK activity, or an increase in the production of IgG2a. Preferably, the enhanced T_{H}1 immune response will include an increase in IgG2a production.

A T_{H}1 immune response may be elicited using a T_{H}1 adjuvant. A T_{H}1 adjuvant will generally elicit increased levels of IgG2a production relative to immunization of the antigen without adjuvant. T_{H}1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred T_{H}1 adjuvants for use in the invention.

A T_{H}2 immune response may include one or more of an increase in one or more of the cytokines associated with a T_{H}2 immune response (such as IL-4, IL-5, IL-6 and IL-10), or an increase in the production of IgG1, IgE, IgA and memory B cells. Preferably, the enhanced T_{H}2 immune response will include an increase in IgG1 production.

A T_{H}2 immune response may be elicited using a T_{H}2 adjuvant. A T_{H}2 adjuvant will generally elicit increased levels of IgG1 production relative to immunization of the antigen without adjuvant. T_{H}2 adjuvants suitable for use in the invention include, for example, mineral containing compositions, oil-emulsions, and ADP-ribosylating toxins and detoxified derivatives thereof. Mineral containing compositions, such as aluminium salts are preferred T_{H}2 adjuvants for use in the invention.

Preferably, the invention includes a composition comprising a combination of a T_{H}1 adjuvant and a T_{H}2 adjuvant. Preferably, such a composition elicits an enhanced T_{H}1 and an enhanced T_{H}2 response, i.e., an increase in the production of both IgG1 and IgG2a production relative to immunization without an adjuvant. Still more preferably, the composition comprising a combination of a T_{H}1 and a T_{H}2 adjuvant elicits an increased T_{H}1 and/or an increased T_{H}2 immune response relative to immunization with a single adjuvant (i.e., relative to immunization with a T_{H}1 adjuvant alone or immunization with a T_{H}2 adjuvant alone).

The immune response may be one or both of a T_{H}1 immune response and a T_{H}2 response. Preferably, immune response provides for one or both of an enhanced T_{H}1 response and an enhanced T_{H}2 response.

The enhanced immune response may be one or both of a systemic and a mucosal immune response. Preferably, the immune response provides for one or both of an enhanced systemic and an enhanced mucosal immune response. Preferably the mucosal immune response is a T_{H}2 immune response. Preferably, the mucosal immune response includes an increase in the production of IgA.

In certain embodiments, treatment or prophylaxis comprises administering a vaccine of the invention in a booster injection or booster dose. C. difficile vaccine boosters provide for re-exposure of a subject's immune system to increase immunity or restore immunity to protective levels.

Thus, in one embodiment, treatment of a patient with CDI comprises administering a vaccine of the invention to boost the patients “primed” immune system. The booster can be administered at any time during the infection, without limitation, at the time that CDI symptoms are detected or observed, or about 1 day, 2 days, 3 days, 4 days, 5 days, 6 days, one week, two weeks or longer after CDI symptoms are detected or observed.

In another embodiment, for a subject previously immunized by vaccination or CDI treatment comprises administering a vaccine of the invention to restore immunity to protective levels. Non-limiting examples of times to administer a booster includes 1, 2, 3, 4, 5, 7, 10, 12, 15, and 20 years after initial immunization. In certain embodiments, the necessity, desirability, or timing of booster administration results from environmental factors, for example risky work environment or a scheduled hospitalization.

The vaccines described herein are also useful to generate neutralizing antibodies which can be used as a passive immune serum to treat or ameliorate the symptoms in patients. A vaccine composition as described above could be administered to an animal such as a horse or a human until a neutralizing antibody response is generated. These neutralizing antibodies can then be harvested, purified, and utilized to treat patients exhibiting symptoms.

According to the invention, the neutralizing antibodies are administered to patients exhibiting disease C. difficile symptoms in an amount effective to neutralize the effect of the pathogen. The neutralizing antibodies can be administered intravenously, intramuscularly, intradermally, subcutaneously, and the like. In certain embodiments, the neutralizing antibodies may be administered with an immunogenic composition of the invention in order to ameliorate the effects of a C. difficile infection while at the same time stimulating a host immune response. In another embodiment, the neutralizing antibody can be administered in conjunction with antibiotic therapy. The amount of neutralizing antibodies typically administered is about 1 mg of antibody to 1000 mg/kg, more preferably about 50-200 mg/kg of body weight.

In certain embodiments, an immunogenic composition or vaccine of the present invention may be administered with another agent for preventing or treating C. difficile infection. The agent can be administered concomitantly with the composition or vaccine, or administered sequentially, e.g., one before the other.

In certain situations, such as during or following treatment with or administration of antibiotics, the natural balance of the gut flora is disturbed. As a result, C. difficile can become more prevalent leading to symptoms of infection. Thus, the immunogenic compositions of the invention may be used in conjunction with antibiotics that treat the underlying condition while the immunogenic composition prophylactically prevents or ameliorates a C. difficile infection.

In certain embodiments of the invention, an effective amount of an immunogenic composition of the invention is administered to a subject together with or separately from an antibiotic at a time which can be before, at the same time, or after administering the antibiotic. The method is effective for use with any antibiotic which is suitable for treating the underlying infection, and more particularly with an antibiotic which is known to be associated with C. difficile AAD. Though any antibiotic can cause antibiotic-associated diarrhea, or one of the more severe C. difficile infection associated conditions, the most common causative agents are ampicillin, clindamycin, cephalosporins such as cefpodoxime, and all fluoroquinolones. These methods are thus particularly suited to treatment regimes incorporating an antibiotic known in the art to be frequently linked to C. difficile AAD. In some embodiments, therefore, a composition of the invention may further comprise an antibiotic, such as an antibiotic listed above.

The invention provides lyophilized vaccine preparation and kits.

Lyophilization, or the process of freeze-drying, is a commonly used technique to remove water in the preparation of dehydrated products. Generally, “freeze-drying” an aqueous composition involves three steps. First, the aqueous composition is frozen under conditions of low temperature. Secondly, the frozen water is removed by sublimation under conditions of reduced pressure and low temperature. At this stage, the composition usually contains about 15% water. Third, the residual water is further removed by desorption under conditions of reduced pressure and higher temperatures. At the end of the lyophilization process, a freeze-dried product, also called a “pastille” or “cake” is produced. The freeze-dried product contains very low residual water (from about 0.5% to about 5% weight/weight) and dry material in an amorphous form. This specific state is qualified as “vitreous”.

However, substantial loss of immunogenic activity of biological ingredients are observed during the preparation stages, such as before and during lyophilization, and also during storage of immunogenic compositions and vaccine compositions. The integrity of biological ingredients must be safeguarded to ensure that the immunization efficiency of immunogenic compositions and vaccine compositions is retained. The immunogenic activity of biological ingredients is measured by the ability to induce and stimulate an immunologic response when administered to a host or subject.

Sugars such as sucrose, raffinose and trehalose have been added in various combinations as stabilizers prior to lyophilization of viruses. A large number of compounds have been tested for their ability to stabilize different vaccines containing live attenuated biological ingredients, in particular viruses. Such compounds include SPGA (sucrose, phosphate, glutamate, and albumin; Bovarnick et al. (1950) J. Bacteriol. 59, 509-522; U.S. Pat. No. 4,000,256), bovine or human serum albumin, alkali metal salts of glutamic acid, aluminum salts, sucrose, gelatin, starch, lactose, sorbitol, Tris-EDTA, casein hydrolysate, sodium and potassium lactobionate, and monometallic or dimetallic alkali metal phosphate. Other compounds include, for example, SPG-NZ amine (e.g. U.S. Pat. No. 3,783,098) and polyvinylpyrrolidone (PVP) mixtures (e.g. U.S. Pat. No. 3,915,794).

Freeze-drying” involves lyophilization and refers to the process by which a suspension is frozen, after which the water is removed by sublimation at low pressure. As used herein, the term “sublimation” refers to a change in the physical properties of a composition, wherein the composition changes directly from a solid state to a gaseous state without becoming a liquid. As used herein, the “T′g value” is defined as the glass transition temperature, which corresponds to the temperature below which the frozen composition becomes vitreous.

A process for freeze-drying an immunogenic suspension or solution according to the invention can comprise the steps of (a) contacting the immunogenic suspension or solution with a stabilizer of the invention, thereby forming a stabilized immunogenic suspension or solution; (b) cooling, at atmospheric pressure, the stabilized immunogenic suspension or solution to a temperature less than about the T′g value of the stabilized immunogenic suspension or solution; (c) drying the stabilized immunogenic suspension or solution (i.e., the primary desiccation or sublimation step) by sublimation of ice at low pressure; and (d) removing excess residual water (i.e., secondary drying or desorption step) by further reducing pressure and increasing the temperature of the stabilized immunogenic suspension or solution.

The cooling step (b) can occur at temperatures of less than about −40° C. (water freezing step). Drying the stabilized immunogenic suspensions or solution by sublimation of ice at low pressure (c) can occur at, for example, pressure lower than or equal to about 200 μbar, whereas a further reduction in pressure can occur at pressures lower than or equal to about 100 μbar. Finally, the temperature of the stabilized immunogenic suspension or solution during the removal of excess residual water (d) occurs at, for example, temperatures between about 20° C. and about 30° C.

The process of freeze-drying can also be performed with an immunogenic suspension or solution comprising live attenuated canine paramyxovirus and at least one active immunogenic component derived from a pathogen other than a paramyxovirus, which is mixed with a stabilizer according to the invention to obtain a freeze-dried stabilized multivalent immunogenic or vaccine composition.

The moisture content of the freeze-dried material can range from about 0.5% to about 5% w/w, preferably from about 0.5% to about 3% w/w, and more preferably from about 1.0% to about 2.6% w/w.

Advantageously, the stabilized immunogenic suspension or solution comprising at least one bulking agent has a high T′g value of between about −36° C. to about −30° C. A high T′g value allows for higher temperatures during the water freezing step of the freezing process and/or the freeze-drying process, thereby decreasing exposure of the live attenuated virus and the active immunogenic component to stress, avoiding substantial loss of activity.

Each step, including water freezing, and its removal during the primary and secondary desiccation, subjects the biological ingredients, such as pathogens, in the immunogenic suspensions or solutions of the invention to mechanical, physical and biochemical shock, which have potentially adverse effects upon the structure, appearance, stability, immunogenicity, infectivity and viability of the pathogens or biological ingredients.

The stabilizers of the invention allow good stability of live attenuated pathogens during the freeze-drying process and during storage. The stability can be calculated by the difference between the infectivity titer before the freeze-drying step, and the infectivity titer after 12 months of storage of the freeze-dried stabilized immunogenic composition or vaccine composition at 4° C. Good stability can advantageously comprise a difference of only 1.2 log₁₀ and preferably of only 1.0 log₁₀. Methods for determining the infectivity titer are well known by those skilled in the art. Also, the stability can be estimated by fitting the log₁₀ titer and the time points of the titration during the period of storage using linear regression calculations and/or algorithms.

Further, the stabilizers of the invention allow for freeze-dried pastilles having a good aspect, in other words, having regular form and uniform color. An irregular form can be characterized by the presence of all or a part of the pastille stuck to the bottom of the recipient and remaining immobile after turning over and shearing (stuck aspect). Also, a pastille having a form of a spool (spooled aspect), or separation of the pastille in two parts, following a horizontal plane (de-duplicated aspect), or a pastille having an aspect of a mousse with irregular holes (spongy aspect), or a pastille having the aspect of foam into the recipient (meringue aspect) have an irregular form and are not accepted.

The stabilized freeze-dried immunogenic compositions or vaccine compositions using a stabilizer according to the present invention and obtained by the process of freeze-drying described above are encompassed in the present invention.

A further aspect of the present invention provides a kit comprising a first container containing the powder vaccine composition of the disclosure, and, optionally, a second container containing a diluent. Or, a kit comprising a first container having the antigenic component and a second container having the other constituents of the powder vaccine composition and optionally a third container having the diluent. Or, a kit simply having a container having the powder vaccine composition. Kits advantageously contain instructions for admixture and administration.

For its use and administration into a subject, the powder vaccine composition can be reconstituted by rehydration with a diluent. The diluent is typically water, such as demineralized or distilled water, but can also comprise physiological solutions or buffers known in the art. The powder vaccine composition when diluted or a reconstituted ready-to-use powder vaccine composition can be administered to an animal by means discussed herein, such as injection through the parenteral or mucosal route, or preferably by oral or ocular administration by spraying. However, administration of dissolved powder vaccine compositions can also comprise intranasal, epicutaneous or topical administration.

The following examples are intended only to further illustrate the invention and are not intended to limit the scope of the invention, which is defined by the appended claims.

### Example 1

A sample of a C. difficile BI/NAP1/027 strain was obtained from American Type Culture Collection, Manassas, Va. (ATCC® BAA-1870). A glycerol stock was used to inoculate a 50 mL Brain Heart Infusion-L-cysteine (BHI-Cys) growth medium starter culture (BHI-BD Bacto #237200; L-Cysteine—Sigma #168149) that was grown overnight at 37° C. under anaerobic conditions for 17 hours. Optical density (OD₆₀₀) reading and contamination tests were performed on the starter culture. 1 L of fresh reduced BHI-Cys medium was inoculated with starter culture to an OD₆₀₀ of 0.1 and incubated at 37° C. anaerobically until an OD₆₀₀ of about 1.0 (5-6 hours of growth) was reached. Cells were harvested by centrifugation (6000 rpm, 20 minutes, 4° C.) and washed three times with 1×PBS. After the last wash, pellets were resuspended in 1×PBS and serial dilutions were prepared to determine the number of colony-forming units (CFU) by plating; cell counts were made using a microscope and hemocytometer, and an OD₆₀₀ reading of the sample. The pellet suspension was split into two equal volumes for use in different inactivating treatments. One portion was adjusted to 1% (v/v) formalin and incubated with rocking at 4° C. for 24 hours. The other portion was heat-treated in a water bath at 80° C. for 30 minutes. Three washes with 1×PBS were performed after each of the inactivation treatments. After the last wash, cells were concentrated by centrifugation and pellets were resuspended in 1×PBS and serial dilutions were prepared to determine reduction in viable counts after inactivation treatment by plating CFU counts. Formalin treatment of processed cells resulted in a greater than 8-log inactivation of C. difficile cells as determined by plating serial dilutions of cells on BHI-Cys-0.1% TA plates and incubation of plates at 37° C. for 48 hours in anaerobic conditions. Heat treatment at 80° C. of processed cells resulted in a greater than 7-log inactivation of C. difficile cells as determined by plating serial dilutions of cells on BHI-Cys-0.1% TA plates and incubation of plates at 37° C. for 48 hours in anaerobic conditions. Cell counts were determined using a microscope and hemocytometer then correlated to an OD₆₀₀ reading of the sample.

The formalin and heat-treated C. difficile cell suspensions were stored at 4° C. On the day of immunization, vaccine candidates were prepared by mixing 10⁸-10⁹ inactivated cells with 0.5 mg/mL of an alum adjuvant (Alhydrogel® sterilized aluminum hydroxide gel; Invivogen). In the combination vaccine treatment groups, 5 μg of a TcdB2 immunogen, i.e., the polypeptide fragment of TcdB2₁₆₅₁₋₂₃₆₆ (SEQ ID NO:3) was added to the same alum-adjuvanted, inactivated whole cell suspension and incubated for at least 2 hours at ambient temperature before administration.

The C. difficile Toxin B immunogen was a recombinant polypeptide expressed in E. coli BL21(DE3) cells (Novagen (EMD-Millipore)) transformed with an expression vector encoding the C-terminal 716 amino acids of TcdB2 toxin, designated CROPB_{HV}. The culture was grown in animal-free media, induced with 0.1 mM Isopropyl β-D-1-thiogalactopyranoside (IPTG) (Teknova, Catalog #13325) and grown overnight at 25° C. The CROPB_{HV} was captured from soluble bacterial lysate by hydrophobic interaction chromatography, followed by ion exchange chromatography, followed by purification on an affinity resin.

### Immunizations

Groups of BALB/c mice (n=5) were immunized by intraperitoneal injection biweekly three times with 10⁸ C. difficile cells inactivated using 1% formalin or by heat-treatment and co-administered with or without 5 μg of CROPB_{HV}. The experiment included one control group of naïve mice receiving no immunizations. A fourth immunization of 10⁹ cells with or without 5 μg CROPB_{HV} was administered to the mice about 2.5 weeks after the 3^{rd} immunization. Seventeen days after the 4^{th} immunization, mice were treated with an antibiotic regimen for 7 days to render them susceptible to infection with C. difficile spores: Briefly, a cocktail of 5 different antibiotics (vancomycin 0.045 mg/ml, metronidazole 0.215 mg/ml, kanamycin 0.4 mg/ml, gentamycin 0.035 mg/ml, and colistin 850 U/ml) was administered to mice in their drinking water with additional gavage of 100 μl of cocktail at least 3 times a week. The mice were returned to regular drinking water for 2-3 days before intraperitoneal administration of clindamycin 10 mg/kg the day before challenge with live bacteria.

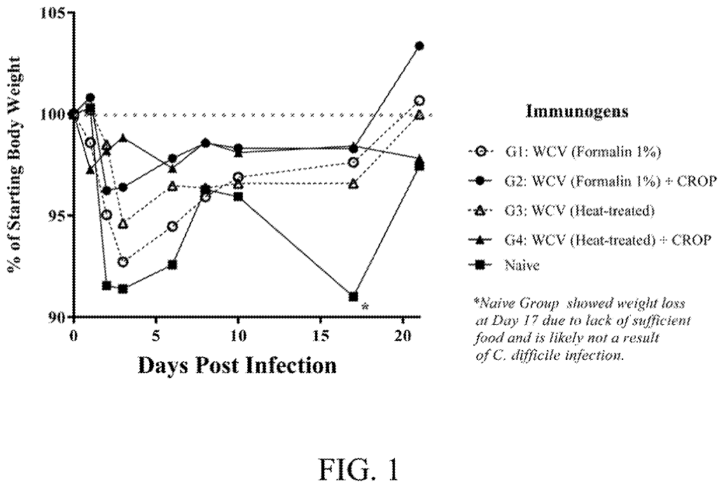

The groups were challenged with 10⁷ C. difficile BI/NAP1/027 spores administered by intragastric gavage. The mice were monitored daily for body weight and other symptoms of C. difficile infection. The results are shown in FIG. 1. FIG. 1 graphs the percentage change in body weight of mice relative to their starting weight prior to infection. Weight loss, a surrogate marker of morbidity of C. difficile infection, was measured at eight time-points over the 20 days following administration of the C. difficile spores. By Day 3 post-challenge with C. difficile, mice immunized with whole cell vaccine (WCV)+CROPB_{HV} showed less weight loss than mice immunized with only WCV or naïve mice. Through Day 10 post-infection, mice immunized with WCV+CROPB_{HV} polypeptide continued to recover body weight more quickly than mice immunized with WCV alone or naïve mice. About 3 weeks post-infection, mice that were administered WCV with or without CROPB_{HV} recovered body weight comparable to pre-infection body weights. Overall, mice immunized with WCV do not lose as much weight and lose it more slowly than naïve mice after C. difficile spore challenge.

Further, we observed that the addition of CROPB_{HV} as a co-immunogen to WCV resulted in a notably greater protective immune response and more rapid recovery from C. difficile spore challenge. IgG antibody titers determined by ELISA for each of the groups presented in Table 2. To measure anti-CROPB_{HV} IgG antibody titers, plates were coated with CROPB_{HV} protein; to measure anti-whole cell IgG antibody titers, plates were coated with inactivated whole cells protein. Pooled antisera were collected on Day 64, 9 days after the fourth immunization, and IgG antibody titers were determined.

TABLE 2

| | Antigen (Dose) | | IgG Antibody Titers - Pooled Antisera^{A} | |
|---|---|---|---|---|
| Group | WC (CFU)^{B} | CROP (μg) | anti-CROPB2 | anti-Whole cell |
| | Formalin | | | |
| G1 | 10⁸/10⁹ | | 10 | 204,800 |
| G2 | 10⁸/10⁹ | 5 | 800,000 | 51,200 |
| | Heat (80° C.) | | | |
| G3 | 10⁸/10⁹ | | 10 | 51,200 |
| G4 | 10⁸/10⁹ | 5 | 1,600,000 | 25,600 |
| Naïve | | | 10 | below detection |

^{A}IgG titers determined by ELISA on Day 64 (9 days after the 4^{th} immunization)
^{B}First three immunizations with 10⁸ cells; 4^{th} immunization with 10⁹ cells

### Example 2

In an experiment evaluating protection against Toxin B_{HV} challenge, CROPB_{HV} immunized mice were completely protected against toxin challenge (10 of 10 mice) whereas 8 of 10 Toxoid B_{HV} immunized mice were protected against toxin challenge (Table 3; FIG. 2). These data demonstrated that CROPB_{HV} is a preferred antigen comparable or better than full-length, inactivated toxin B_{HV}.

TABLE 3

| | | No. of | Toxic Challenge | | TTD (hr) |
|---|---|---|---|---|---|
| Group | Vaccine | Mice | Dose | % Survival | (GMT) |
| G1 | CROPB2 + AlOH | 10 | 100 ng B2 Toxin | 100 | 166 |
| G2 | ToxoidB + AlOH | 10 | 100 ng B2 Toxin | 80 | 112.03 |
| G3 | PBS | 10 | 100 ng B2 Toxin | 10 | 8.1 |
| G4 | PBS | 10 | NONE | 100 | 166 |

### Example 3

A sample of a non-toxigenic C. difficile strain was obtained from Dr. Dale Gerding (Hines Veteran Affairs Hospital, Chicago, Ill.). A glycerol stock is used to inoculate a 50 mL Brain Heart Infusion-L-cysteine (BHI-Cys) growth medium starter culture (BHI—BD Bacto #237200; L-Cysteine—Sigma #168149) that is grown overnight at 37° C. under anaerobic conditions for 17 hours. Optical density (OD₆₀₀) reading and contamination tests are performed on the starter culture. 1 L of fresh reduced BHI-Cys medium is inoculated with starter culture to an OD₆₀₀ of 0.1 and incubated at 37° C. anaerobically until an OD₆₀₀ of about 1.0 (5-6 hours of growth) is reached. Cells are harvested by centrifugation (6000 rpm, 20 minutes, 4° C.) and washed three times with 1×PBS. After the last wash, pellets are resuspended in 1×PBS and serial dilutions prepared to determine the number of colony-forming units (CFU) by plating; cell counts are made using a microscope and hemocytometer, and an OD₆₀₀ reading of the sample. The pellet suspension is split into two equal volumes for use in different inactivating treatments. One portion is adjusted to 1% (v/v) formalin and incubated with rocking at 4° C. for 24 hours. The other portion is heat-treated in a water bath at 80° C. for 30 minutes. Three washes with 1×PBS are performed after each of the inactivation treatments. After the last wash, cells are concentrated by centrifugation, pellets resuspended in 1×PBS and serial dilutions prepared to determine reduction in viable counts after inactivation treatment by plating CFU counts. Formalin treatment of processed cells typically results in a greater than 8-log inactivation of C. difficile cells as determined by plating serial dilutions of cells on BHI-Cys-0.1% TA plates and incubation of plates at 37° C. for 48 hours in anaerobic conditions. Heat treatment at 80° C. of processed cells typically results in a greater than 7-log inactivation of C. difficile cells as determined by plating serial dilutions of cells on BHI-Cys-0.1% TA plates and incubation of plates at 37° C. for 48 hours in anaerobic conditions. Cell counts are determined using a microscope and hemocytometer then correlated to an OD₆₀₀ reading of the sample.

The formalin and heat-treated C. difficile cell suspensions are stored at 4° C. On the day of immunization, vaccine candidates are prepared by mixing 10⁸-10⁹ inactivated cells with 0.5 mg/mL of an alum adjuvant (Alhydrogel® sterilized aluminum hydroxide gel; Invivogen). In the combination vaccine treatment groups, 5 μg of a TcdB2 immunogen, i.e., the polypeptide fragment of TcdB2₁₆₅₁₋₂₃₆₆ (SEQ ID NO:3) are added to the same alum-adjuvanted, inactivated whole cell suspension and incubated for at least 2 hours at ambient temperature before administration.

The C. difficile Toxin B immunogen is typically a recombinant polypeptide expressed in E. coli BL21(DE3) cells (Novagen (EMD-Millipore)) transformed with an expression vector encoding, for example, the C-terminal 716 amino acids of TcdB2 toxin. The culture is grown in animal-free media, induced with 0.1 mM Isopropyl β-D-1-thiogalactopyranoside (IPTG) (Teknova, Catalog #13325) and grown overnight at 25° C. The C-terminal polypeptide fragment of TcdB2 can be captured from soluble bacterial lysate by hydrophobic interaction chromatography, followed by ion exchange chromatography, followed by purification on an affinity resin.

### Immunizations

Groups of BALB/c mice (n=5) are immunized by intraperitoneal injection biweekly three times with 10⁸ or 10⁹ C. difficile cells inactivated using 1% formalin or by heat-treatment and co-administered with or without 5 μg of CROPB_{HV}. The experiment includes one control group of naïve mice receiving no immunizations. Seventeen days after the 3^{rd} immunization, mice are treated with an antibiotic regimen for 7 days to render them susceptible to infection with C. difficile spores: Briefly, a cocktail of 5 different antibiotics (vancomycin 0.045 mg/ml, metronidazole 0.215 mg/ml, kanamycin 0.4 mg/ml, gentamycin 0.035 mg/ml, and colistin 850 U/ml) is administered to mice in their drinking water with additional gavage of 100 μl of cocktail at least 3 times a week. The mice are returned to regular drinking water for 2-3 days before intraperitoneal administration of clindamycin 10 mg/kg the day before challenge with live bacteria. The groups are challenged with 10⁷ C. difficile BI/NAP1/027 spores administered by intragastric gavage. The mice are monitored daily for body weight and other symptoms of C. difficile infection.

Overall, mice immunized with WCV lose less weight and lose it more slowly than naïve mice after C. difficile spore challenge. Further, the addition of TcdB2 CROP polypeptide as a co-immunogen to WCV results in a notably greater protective immune response and more rapid recovery from C. difficile spore challenge.

### Example 4

A sample of a C. difficile BI/NAP1/027 strain was obtained from American Type Culture Collection, Manassas, Va. (ATCC® BAA-1870). A glycerol stock was used to inoculate a 50 mL Brain Heart Infusion-L-cysteine (BHI-Cys) growth medium starter culture (BHI—BD Bacto #237200; L-Cysteine—Sigma #168149) that was grown overnight at 37° C. under anaerobic conditions for 17 hours. Optical density (OD₆₀₀) reading and contamination tests were performed on the starter culture. 1 L of fresh reduced BHI-Cys medium was inoculated with starter culture to an OD₆₀₀ of 0.1 and incubated at 37° C. anaerobically until an OD₆₀₀ of about 1.0 (5-6 hours of growth) was reached. Cells were harvested by centrifugation (6000 rpm, 20 minutes, 4° C.) and the cell paste was stored at −80° C.

To prepare the cell surface extract, the cell paste was thawed at ambient temperature then was reconstituted in 80 mL of 0.5% sodium deoxycholate. The cell solution was then placed in a shaking incubator (225 rpm) set at 60° C. and the solution was incubated for 16-24 hours. The cell solution was removed from the incubator and cooled to ambient temperature prior to centrifugation at 6000 rpm at 4° C. to remove cells. The supernatant containing the cell surface components was saved for further processing. The supernatant underwent tangential flow filtration using filter membranes with a 5000 Da molecular weight cutoff to first wash away the deoxycholate then to concentrate the supernatant 5-10-fold. The concentrated supernatant was designated the cell surface extract (CSE). The CSE was analyzed to determine the protein content using a standard assay and polysaccharide content using an anthrone assay that measures the presence of carbohydrate.

The C. difficile Toxin B immunogen was a recombinant polypeptide expressed in E. coli BL21(DE3) cells (Novagen (EMD-Millipore)) transformed with an expression vector encoding the C-terminal 716 amino acids of TcdB2 toxin, designated CROPB_{HV}. The culture was grown in animal-free media, induced with 0.1 mM Isopropyl β-D-1-thiogalactopyranoside (IPTG) (Teknova, Catalog #13325) and grown overnight at 25° C. The C-terminal polypeptide fragment of TcdB2 was captured from soluble bacterial lysate by hydrophobic interaction chromatography, followed by ion exchange chromatography, followed by purification on an affinity resin. The Toxin A immunogen was purified in a similar manner.

### Immunizations

Groups of BALB/c mice (n=5) are immunized by intraperitoneal injection biweekly three times with CSE, containing 25 μg of polysaccharide, administered with or without 5 μg of CROPB_{HV} or 5 μg TcdA polypeptide. The experiment includes one control group of naïve mice receiving no immunizations. Seventeen days after the 3^{rd} immunization, mice are treated with an antibiotic regimen for 7 days to render them susceptible to infection with C. difficile spores: Briefly, a cocktail of 5 different antibiotics (vancomycin 0.045 mg/ml, metronidazole 0.215 mg/ml, kanamycin 0.4 mg/ml, gentamycin 0.035 mg/ml, and colistin 850 U/ml) is administered to mice in their drinking water with additional gavage of 100 μl of cocktail at least 3 times a week. The mice are returned to regular drinking water for 2-3 days before intraperitoneal administration of clindamycin 10 mg/kg the day before challenge with live bacteria. The groups are challenged with 10⁷ C. difficile BI/NAP1/027 spores administered by intragastric gavage. The mice are monitored daily for body weight and other symptoms of C. difficile infection. The results are expected to show that immunization with CSE plus Toxin B antigen is more protective (quicker recovery of weight and fewer disease symptoms) than mice immunized with only CSE which in turn will show a quicker recovery and fewer symptoms of infection than the naïve group.

It will be appreciated that the above descriptions are illustrative and not limiting of the present invention. Additional embodiments are possible and will suggest themselves to those skilled in the art upon consideration of the full scope of the present disclosure and the invention as defined in the appended claims.

### Index of DNA/Amino Acid Sequences

A sequence listing is included herewith setting forth the polypeptide amino acid sequences identified herein, including the following:

SEQ ID NO:1—amino acid sequence of C. difficile Toxin B of VPI 10463

SEQ ID NO:2—amino acid sequence of C. difficile Toxin B of a NAP1/027 strain

SEQ ID NO:3—amino acid sequence of C. difficile TcdB2₁₆₅₁₋₂₃₆₆

SEQ ID NO:4—amino acid sequence of C. difficile Toxin A of VPI 10463

SEQ ID NO:5—amino acid sequence of C. difficile Toxin A of a NAP1/027 strain

SEQ ID NO:6—amino acid sequence of a CROPs region of Toxin A from a C. difficile NAP1/027 strain.

| SEQ ID NO: | |
|---|---|
| 1 | MSLVNRKQLE KMANVRFRTQ EDEYVAILDA LEEYHNMSEN |
| | TVVEKYLKLK |
| | DINSLTDIYI DTYKKSGRNK ALKKFKEYLV TEVLELKNNN |
| | LTPVEKNLHF |
| | VWIGGQINDT AINYINQWKD VNSDYNVNVF YDSNAFLINT |
| | LKKTVVESAI |
| | NDTLESFREN LNDPRFDYNK FFRKRMEIIY DKQKNFINYY |
| | KAQREENPEL |
| | IIDDIVKTYL SNEYSKEIDE LNTYIEESLN KITQNSGNDV RNFEEFKNGE |
| | SFNLYEQELV ERWNLAAASD ILRISALKEI GGMYLDVDML |
| | PGIQPDLFES |
| | IEKPSSVTVD FWEMTKLEAI MKYKEYIPEY TSEHFDMLDE |
| | EVQSSFESVL |
| | ASKSDKSEIF SSLGDMEASP LEVKIAFNSK GIINQGLISV KDSYCSNLIV |
| | KQIENRYKIL NNSLNPAISE DNDFNTTTNT FIDSIMAEAN |
| | ADNGRFMMEL |
| | GKYLRVGFFP DVKTTINLSG PEAYAAAYQD LLMFKEGSMN |
| | IHLIEADLRN |
| | FEISKTNISQ STEQEMASLW SFDDARAKAQ FEEYKRNYFE |
| | GSLGEDDNLD |
| | FSQNIVVDKE YLLEKISSLA RSSERGYIHY IVQLQGDKIS |
| | YEAACNLFAK |
| | TPYDSVLFQK NIEDSEIAYY YNPGDGEIQE IDKYKIPSII SDRPKIKLTF |
| | IGHGKDEFNT DIFAGFDVDS LSTEIEAAID LAKEDISPKS IEINLLGCNM |
| | FSYSINVEET YPGKLLLKVK DKISELMPSI SQDSIIVSAN QYEVRINSEG |
| | RRELLDHSGE WINKEESIIK DISSKEYISF NPKENKITVK SKNLPELSTL |
| | LQEIRNNSNS SDIELEEKVM LTECEINVIS NIDTQIVEER IEEAKNLTSD |
| | SINYIKDEFK LIESISDALC DLKQQNELED SHFISFEDIS ETDEGFSIRF |
| | INKETGESIF VETEKTIFSE YANHITEEIS KIKGTIFDTV NGKLVKKVNL |
| | DTTHEVNTLN AAFFIQSLIE YNSSKESLSN LSVAMKVQVY |
| | AQLFSTGLNT |
| | ITDAAKVVEL VSTALDETID LLPTLSEGLP IIATIIDGVS LGAAIKELSE |
| | TSDPLLRQEI EAKIGIMAVN LTTATTAIIT SSLGIASGFS ILLVPLAGIS |
| | AGIPSLVNNE LVLRDKATKV VDYFKHVSLV ETEGVFTLLD |
| | DKIMMPQDDL |
| | VISEIDFNNN SIVLGKCEIW RMEGGSGHTV TDDIDHFFSA PSITYREPHL |
| | SIYDVLEVQK EELDLSKDLM VLPNAPNRVF AWETGWTPGL |
| | RSLENDGTKL |
| | LDRIRDNYEG EFYWRYFAFI ADALITTLKP RYEDTNIRIN LDSNTRSFIV |
| | PIITTEYIRE KLSYSFYGSG GTYALSLSQY NMGINIELSE SDVWIIDVDN |
| | VVRDVTIESD KIKKGDLIEG ILSTLSIEEN KIILNSHEIN FSGEVNGSNG |
| | FVSLTFSILE GINAIIEVDL LSKSYKLLIS GELKILMLNS NHIQQKIDYI |
| | GFNSELQKNI PYSFVDSEGK ENGFINGSTK EGLFVSELPD |
| | VVLISKVYMD |
| | DSKPSFGYYS NNLKDVKVIT KDNVNILTGY YLKDDIKISL |
| | SLTLQDEKTI |
| | KLNSVHLDES GVAEILKFMN RKGNTNTSDS LMSFLESMNI |
| | KSIFVNFLQS |
| | NIKFILDANF IISGTTSIGQ FEFICDENDN IQPYFIKENT LETNYTLYVG |
| | NRQNMIVEPN YDLDDSGDIS STVINFSQKY LYGIDSCVNK |
| | VVISPNIYTD |
| | EINITPVYET NNTYPEVIVL DANYINEKIN VNINDLSIRY VWSNDGNDFI |
| | LMSTSEENKV SQVKIRFVNV FKDKTLANKL SFNFSDKQDV |
| | PVSEIILSFT |
| | PSYYEDGLIG YDLGLVSLYN EKFYINNEGM MVSGLIYIND |
| | SLYYFKPPVN |
| | NLITGFVTVG DDKYYFNPIN GGAASIGETI IDDKNYYFNQ |
| | SGVLQTGVFS |
| | TEDGFKYFAP ANTLDENLEG EAIDFTGKLI IDENIYYFDD |
| | NYRGAVEWKE |
| | LDGEMHYFSP ETGKAFKGLN QIGDYKYYFN SDGVMQKGFV |
| | SINDNKHYFD |
| | DSGVMKVGYT EIDGKHFYFA ENGEMQIGVF NTEDGFKYFA |
| | HHNEDLGNEE |
| | GEEISYSGIL NENNKIYYFD DSFTAVVGWK DLEDGSKYYF |
| | DEDTAEAYIG |
| | LSLINDGQYY FNDDGIMQVG FVTINDKVFY FSDSGIIESG |
| | VQNIDDNYFY |
| | IDDNGIVQIG VFDTSDGYKY FAPANTVNDN IYGQAVEYSG |
| | LVRVGEDVYY |
| | FGETYTIETG WIYDMENESD KYYFNPETKK ACKGINLIDD |
| | IKYYFDEKGI |
| | MRTGLISFEN NNYYFNENGE MQFGYINIED KMFYFGEDGV |
| | MQIGVFNTPD |
| | GFKYFAHQNT LDENFEGESI NYTGWLDLDE KRYYFTDEYI |
| | AATGSVIIDG |
| | EEYYFDPDTA QLVISE |
| 2 | MSLVNRKQLE KMANVRFRVQ EDEYVAILDA LEEYHNMSEN |
| | TVVEKYLKLK |
| | DINSLTDIYI DTYKKSGRNK ALKKFKEYLV TEVLELKNNN |
| | LTPVEKNLHF |
| | VWIGGQINDT AINYINQWKD VNSDYNVNVF YDSNAFLINT |
| | LKKTIVESAT |
| | NDTLESFREN LNDPRFDYNK FYRKRMEIIY DKQKNFINYY |
| | KTQREENPDL |
| | IIDDIVKIYL SNEYSKDIDE LNSYIEESLN KVTENSGNDV RNFEEFKGGE |
| | SFKLYEQELV ERWNLAAASD ILRISALKEV GGVYLDVDML |
| | PGIQPDLFES |
| | IEKPSSVTVD FWEMVKLEAI MKYKEYIPGY TSEHFDMLDE |
| | EVQSSFESVL |
| | ASKSDKSEIF SSLGDMEASP LEVKIAFNSK GIINQGLISV KDSYCSNLIV |
| | KQIENRYKIL NNSLNPAISE DNDFNTTTNA FIDSIMAEAN |
| | ADNGRFMMEL |
| | GKYLRVGFFP DVKTTINLSG PEAYAAAYQD LLMFKEGSMN |
| | IHLIEADLRN |
| | FEISKTNISQ STEQEMASLW SFDDARAKAQ FEEYKKNYFE |
| | GSLGEDDNLD |
| | FSQNTVVDKE YLLEKISSLA RSSERGYIHY IVQLQGDKIS |
| | YEAACNLFAK |
| | TPYDSVLFQK NIEDSEIAYY YNPGDGEIQE IDKYKIPSII SDRPKIKLTF |
| | IGHGKDEFNT DIFAGLDVDS LSTEIETAID LAKEDISPKS IEINLLGCNM |
| | FSYSVNVEET YPGKLLLRVK DKVSELMPSI SQDSIIVSAN |
| | QYEVRINSEG |
| | RRELLDHSGE WINKEESIIK DISSKEYISF NPKENKIIVK SKNLPELSTL |
| | LQEIRNNSNS SDIELEEKVM LAECEINVIS NIDTQVVEGR IEEAKSLTSD |
| | SINYIKNEFK LIESISDALY DLKQQNELEE SHFISFEDIL ETDEGFSIRF |
| | IDKETGESIF VETEKAIFSE YANHITEEIS KIKGTIFDTV NGKLVKKVNL |
| | DATHEVNTLN AAFFIQSLIE YNSSKESLSN LSVAMKVQVY |
| | AQLFSTGLNT |
| | ITDAAKVVEL VSTALDETID LLPTLSEGLP VIATIIDGVS LGAAIKELSE |
| | TSDPLLRQEI EAKIGIMAVN LTAATTAIIT SSLGIASGFS ILLVPLAGIS |
| | AGIPSLVNNE LILRDKATKV VDYFSHISLA ESEGAFTSLD |
| | DKIMMPQDDL |
| | VISEIDFNNN SITLGKCEIW RMEGGSGHTV TDDIDHFFSA PSITYREPHL |
| | SIYDVLEVQK EELDLSKDLM VLPNAPNRVF AWETGWTPGL |
| | RSLENDGTKL |
| | LDRIRDNYEG EFYWRYFAFI ADALITTLKP RYEDTNIRIN LDSNTRSFIV |
| | PVITTEYIRE KLSYSFYGSG GTYALSLSQY NMNINIELNE |
| | NDTWVIDVDN |
| | VVRDVTIESD KIKKGDLIEN ILSKLSIEDN KIILDNHEIN FSGTLNGGNG |
| | FVSLTFSILE GINAVIEVDL LSKSYKVLIS GELKTLMANS NSVQQKIDYI |
| | GLNSELQKNI PYSFMDDKGK ENGFINCSTK EGLFVSELSD |
| | VVLISKVYMD |
| | NSKPLFGYCS NDLKDVKVIT KDDVIILTGY YLKDDIKISL SFTIQDENTI |
| | KLNGVYLDEN GVAEILKFMN KKGSTNTSDS LMSFLESMNI |
| | KSIFINSLQS |
| | NTKLILDTNF IISGTTSIGQ FEFICDKDNN IQPYFIKENT LETKYTLYVG |
| | NRQNMIVEPN YDLDDSGDIS STVINFSQKY LYGIDSCVNK VIISPNIYTD |
| | EINITPIYEA NNTYPEVIVL DTNYISEKIN ININDLSIRY VWSNDGSDFI |
| | LMSTDEENKV SQVKIRFTNV FKGNTISDKI SFNFSDKQDV SINKVISTFT |
| | PSYYVEGLLN YDLGLISLYN EKFYINNEGM MVSGLVYIND |
| | SLYYFKPPIK |
| | NLITGFTTIG DDKYYFNPDN GGAASVGETI IDGKNYYFSQ |
| | NGVLQTGVFS |
| | TEDGFKYFAP ADTLDENLEG EAIDFTGKLT IDENVYYFGD |
| | NYRAAIEWQT |
| | LDDEVYYFST DTGRAFKGLN QIGDDKEYEN SDGIMQKGFV |
| | NINDKTFYFD |
| | DSGVMKSGYT EIDGKYFYFA ENGEMQIGVF NTADGFKYFA |
| | HHDEDLGNEE |
| | GEALSYSGIL NENNKIYYFD DSFTAVVGWK DLEDGSKYYF |
| | DEDTAEAYIG |
| | ISIINDGKYY FNDSGIMQIG FVTINNEVFY FSDSGIVESG MQNIDDNYFY |
| | IDENGLVQIG VFDTSDGYKY FAPANTVNDN IYGQAVEYSG |
| | LVRVGEDVYY |
| | FGETYTIETG WIYDMENESD KYYFDPETKK AYKGINVIDD |
| | IKYYFDENGI |
| | MRTGLITFED NHYYFNEDGI MQYGYLNIED KTFYFSEDGI |
| | MQIGVFNTPD |
| | GFKYFAHQNT LDENFEGESI NYTGWLDLDE KRYYFTDEYI |
| | AATGSVIIDG |
| | EEYYFDPDTA QLVISE |
| 3 | NRQNMIVEPN YDLDDSGDIS STVINFSQKY LYGIDSCVNK VIISPNIYTD |
| | EINITPIYEA NNTYPEVIVL DTNYISEKIN ININDLSIRY VWSNDGSDFI |
| | LMSTDEENKV SQVKIRFTNV FKGNTISDKI SFNFSDKQDV SINKVISTFT |
| | PSYYVEGLLN YDLGLISLYN EKFYINNEGM MVSGLVYIND |
| | SLYYFKPPIK |
| | NLITGFTTIG DDKYYFNPDN GGAASVGETI IDGKNYYFSQ |
| | NGVLQTGVFS |
| | TEDGFKYFAP ADTLDENLEG EAIDFTGKLT IDENVYYFGD |
| | NYRAAIEWQT |
| | LDDEVYYFST DTGRAFKGLN QIGDDKFYFN SDGIMQKGFV |
| | NINDKTFYFD |
| | DSGVMKSGYT EIDGKYFYFA ENGEMQIGVF NTADGFKYFA |
| | HHDEDLGNEE |
| | GEALSYSGIL NFNNKIYYFD DSFTAVVGWK DLEDGSKYYF |
| | DEDTAEAYIG |
| | ISIINDGKYY FNDSGIMQIG FVTINNEVFY FSDSGIVESG MQNIDDNYFY |
| | IDENGLVQIG VFDTSDGYKY FAPANTVNDN IYGQAVEYSG |
| | LVRVGEDVYY |
| | FGETYTIETG WIYDMENESD KYYFDPETKK AYKGINVIDD |
| | IKYYFDENGI |
| | MRTGLITFED NHYYFNEDGI MQYGYLNIED KTFYFSEDGI |
| | MQIGVFNTPD |
| | GFKYFAHQNT LDENFEGESI NYTGWLDLDE KRYYFTDEYI |
| | AATGSVIIDG |
| | EEYYFDPDTA QLVISE |
| 4 | MSLISKEELI KLAYSIRPRE NEYKTILTNL DEYNKLTTNN |
| | NENKYLQLKK |
| | LNESIDVFMN KYKTSSRNRA LSNLKKDILK EVILIKNSNT |
| | SPVEKNLHFV |
| | WIGGEVSDIA LEYIKQWADI NAEYNIKLWY DSEAFLVNTL |
| | KKAIVESSTT |
| | EALQLLEEEI QNPQFDNMKF YKKRMEFIYD RQKRFINYYK |
| | SQINKPTVPT |
| | IDDIIKSHLV SEYNRDETVL ESYRTNSLRK INSNHGIDIR ANSLFTEQEL |
| | LNIYSQELLN RGNLAAASDI VRLLALKNFG GVYLDVDMLP |
| | GIHSDLFKTI |
| | SRPSSIGLDR WEMIKLEAIM KYKKYINNYT SENFDKLDQQ |
| | LKDNFKLIIE |
| | SKSEKSEIFS KLENLNVSDL EIKIAFALGS VINQALISKQ GSYLTNLVIE |
| | QVKNRYQFLN QHLNPAIESD NNFTDTTKIF HDSLFNSATA |
| | ENSMELTKIA |
| | PYLQVGFMPE ARSTISLSGP GAYASAYYDF INLQENTIEK |
| | TLKASDLIEF |
| | KFPENNLSQL TEQEINSLWS FDQASAKYQF EKYVRDYTGG |
| | SLSEDNGVDF |
| | NKNTALDKNY LLNNKIPSNN VEEAGSKNYV HYIIQLQGDD |
| | ISYEATCNLF |
| | SKNPKNSIII QRNMNESAKS YFLSDDGESI LELNKYRIPE |
| | RLKNKEKVKV |
| | TFIGHGKDEF NTSEFARLSV DSLSNEISSF LDTIKLDISP KNVEVNLLGC |
| | NMESYDFNVE ETYPGKLLLS IMDKITSTLP DVNKNSITIG |
| | ANQYEVRINS |
| | EGRKELLAHS GKWINKEEAI MSDLSSKEYI FFDSIDNKLK |
| | AKSKNIPGLA |
| | SISEDIKTLL LDASVSPDTK FILNNLKLNI ESSIGDYIYY EKLEPVKNII |
| | HNSIDDLIDE FNLLENVSDE LYELKKLNNL DEKYLISFED |
| | ISKNNSTYSV |
| | RFINKSNGES VYVETEKEIF SKYSEHITKE ISTIKNSIIT DVNGNLLDNI |
| | QLDHTSQVNT LNAAFFIQSL IDYSSNKDVL NDLSTSVKVQ |
| | LYAQLFSTGL |
| | NTIYDSIQLV NLISNAVNDT INVLPTITEG IPIVSTILDG INLGAAIKEL |
| | LDEHDPLLKK ELEAKVGVLA INMSLSIAAT VASIVGIGAE VTIFLLPIAG |
| | ISAGIPSLVN NELILHDKAT SVVNYFNHLS ESKKYGPLKT EDDKILVPID |
| | DLVISEIDFN NNSIKLGTCN ILAMEGGSGH TVTGNIDHFF SSPSISSHIP |
| | SLSIYSAIGI ETENLDFSKK IMMLPNAPSR VFWWETGAVP |
| | GLRSLENDGT |
| | RLLDSIRDLY PGKFYWRFYA FFDYAITTLK PVYEDTNIKI |
| | KLDKDTRNFI |
| | MPTITTNEIR NKLSYSFDGA GGTYSLLLSS YPISTNINLS KDDLWIFNID |
| | NEVREISIEN GTIKKGKLIK DVLSKIDINK NKLIIGNQTI DFSGDIDNKD |
| | RYIFLTCELD DKISLIIEIN LVAKSYSLLL SGDKNYLISN LSNTIEKINT |
| | LGLDSKNIAY NYTDESNNKY FGAISKTSQK SIIHYKKDSK |
| | NILEFYNDST |
| | LEFNSKDFIA EDINVFMKDD INTITGKYYV DNNTDKSIDF |
| | SISLVSKNQV |
| | KVNGLYLNES VYSSYLDFVK NSDGHHNTSN FMNLFLDNIS |
| | FWKLFGFENI |
| | NFVIDKYFTL VGKTNLGYVE FICDNNKNID IYFGEWKTSS |
| | SKSTIFSGNG |
| | RNVVVEPIYN PDTGEDISTS LDFSYEPLYG IDRYINKVLI APDLYTSLIN |
| | INTNYYSNEY YPEIIVLNPN TFHKKVNINL DSSSFEYKWS TEGSDFILVR |
| | YLEESNKKIL QKIRIKGILS NTQSFNKMSI DFKDIKKLSL GYIMSNFKSF |
| | NSENELDRDH LGFKIIDNKT YYYDEDSKLV KGLININNSL |
| | FYFDPIEFNL |
| | VTGWQTINGK KYYFDINTGA ALTSYKIING KHFYFNNDGV |
| | MQLGVFKGPD |
| | GFEYFAPANT QNNNIEGQAI VYQSKFLTLN GKKYYFDNNS |
| | KAVTGWRIIN |
| | NEKYYFNPNN AIAAVGLQVI DNNKYYFNPD TAIISKGWQT |
| | VNGSRYYFDT |
| | DTAIAFNGYK TIDGKHFYFD SDCVVKIGVF STSNGFEYFA |
| | PANTYNNNIE |
| | GQAIVYQSKF LTLNGKKYYF DNNSKAVTGW QTIDSKKYYF |
| | NTNTAEAATG |
| | WQTIDGKKYY FNTNTAEAAT GWQTIDGKKY YFNTNTAIAS |
| | TGYTIINGKH |
| | FYFNTDGIMQ IGVFKGPNGF EYFAPANTDA NNIEGQAILY |
| | QNEFLTLNGK |
| | KYYFGSDSKA VTGWRIINNK KYYFNPNNAI AAIHLCTINN |
| | DKYYFSYDGI |
| | LQNGYITIER NNFYFDANNE SKMVTGVFKG PNGFEYFAPA |
| | NTHNNNIEGQ |
| | AIVYQNKFLT LNGKKYYFDN DSKAVTGWQT IDGKKYYFNL |
| | NTAEAATGWQ |
| | TIDGKKYYFN LNTAEAATGW QTIDGKKYYF NTNTFIASTG |
| | YTSINGKHFY |
| | FNTDGIMQIG VFKGPNGFEY FAPANTDANN IEGQAILYQN |
| | KFLTLNGKKY |
| | YFGSDSKAVT GLRTIDGKKY YFNTNTAVAV TGWQTINGKK |
| | YYFNTNTSIA |
| | STGYTIISGK HFYFNTDGIM QIGVFKGPDG FEYFAPANTD |
| | ANNIEGQAIR |
| | YQNRFLYLHD NIYYFGNNSK AATGWVTIDG NRYYFEPNTA |
| | MGANGYKTID |
| | NKNFYFRNGL PQIGVFKGSN GFEYFAPANT DANNIEGQAI |
| | RYQNRFLEILL |
| | GKIYYFGNNS KAVTGWQTIN GKVYYFMPDT AMAAAGGLFE |
| | IDGVIYFFGV |
| | DGVKAPGIYG |
| 5 | MSLISKEELI KLAYSIRPRE NEYKTILTNL DEYNKLTTNN |
| | NENKYLQLKK |
| | LNESIDVFMN KYKNSSRNRA LSNLKKDILK EVILIKNSNT |
| | SPVEKNLHFV |
| | WIGGEVSDIA LEYIKQWADI NAEYNIKLWY DSEAFLVNTL |
| | KKAIVESSTT |
| | EALQLLEEEI QNPQFDNMKF YKKRMEFIYD RQKRFINYYK |
| | SQINKPTVPT |
| | IDDIIKSHLV SEYNRDETLL ESYRTNSLRK INSNHGIDIR ANSLFTEQEL |
| | LNIYSQELLN RGNLAAASDI VRLLALKNFG GVYLDVDMLP |
| | GIHSDLFKTI |
| | PRPSSIGLDR WEMIKLEAIM KYKKYINNYT SENFDKLDQQ |
| | LKDNFKLIIE |
| | SKSEKSEIFS KLENLNVSDL EIKIAFALGS VINQALISKQ GSYLTNLVIE |
| | QVKNRYQFLN QHLNPAIESD NNFTDTTKIF HDSLFNSATA |
| | ENSMELTKIA |
| | PYLQVGFMPE ARSTISLSGP GAYASAYYDF INLQENTIEK |
| | TLKASDLIEF |
| | KFPENNLSQL TEQEINSLWS FDQASAKYQF EKYVRDYTGG |
| | SLSEDNGVDF |
| | NKNTALDKNY LLNNKIPSNN VEEAGSKNYV HYIIQLQGDD |
| | ISYEATCNLF |
| | SKNPKNSIII QRNMNESAKS YFLSDDGESI LELNKYRIPE |
| | RLKNKEKVKV |
| | TFIGHGKDEF NTSEFARLSV DSLSNEISSF LDTIKLDISP KNVEVNLLGC |
| | NMFSYDFNVE ETYPGKLLLS IMDKITSTLP DVNKDSITIG |
| | ANQYEVRINS |
| | EGRKELLAHS GKWINKEEAI MSDLSSKEYI FFDSIDNKLK |
| | AKSKNIPGLA |
| | SISEDIKTLL LDASVSPDTK FILNNLKLNI ESSIGDYIYY EKLEPVKNII |
| | HNSIDDLIDE FNLLENVSDE LYELKKLNNL DEKYLISFED |
| | ISKNNSTYSV |
| | RFINKSNGES VYVETEKEIF SKYSEHITKE ISTIKNSIIT DVNGNLLDNI |
| | QLDHTSQVNT LNAAFFIQSL IDYSSNKDVL NDLSTSVKVQ |
| | LYAQLFSTGL |
| | NTIYDSIQLV NLISNAVNDT INVLPTITEG IPIVSTILDG INLGAAIKEL |
| | LDEHDPLLKK ELEAKVGVLA INMSLSIAAT VASIVGIGAE VTIFLLPIAG |
| | ISAGIPSLVN NELILHDKAT SVVNYFNHLS ESKEYGPLKT EDDKILVPID |
| | DLVISEIDFN NNSIKLGTCN ILAMEGGSGH TVTGNIDHFF SSPYISSHIP |
| | SLSVYSAIGI KTENLDFSKK IMMLPNAPSR VFWWETGAVP |
| | GLRSLENNGT |
| | KLLDSIRDLY PGKFYWRFYA FFDYAITTLK PVYEDTNTKI |
| | KLDKDTRNFI |
| | MPTITTDEIR NKLSYSFDGA GGTYSLLLSS YPISMNINLS KDDLWIFNID |
| | NEVREISIEN GTIKKGNLIE DVLSKIDINK NKLIIGNQTI DFSGDIDNKD |
| | RYIFLTCELD DKISLIIEIN LVAKSYSLLL SGDKNYLISN LSNTIEKINT |
| | LGLDSKNIAY NYTDESNNKY FGAISKTSQK SIIHYKKDSK |
| | NILEFYNGST |
| | LEFNSKDFIA EDINVFMKDD INTITGKYYV DNNTDKSIDF |
| | SISLVSKNQV |
| | KVNGLYLNES VYSSYLDFVK NSDGHHNTSN FMNLFLNNIS |
| | FWKLFGFENI |
| | NFVIDKYFTL VGKTNLGYVE FICDNNKNID IYFGEWKTSS |
| | SKSTIFSGNG |
| | RNVVVEPIYN PDTGEDISTS LDFSYEPLYG IDRYINKVLI APDLYTSLIN |
| | INTNYYSNEY YPEIIVLNPN TFHKKVNINL DSSSFEYKWS TEGSDFILVR |
| | YLEESNKKIL QKIRIKGILS NTQSFNKMSI DFKDIKKLSL GYIMSNFKSF |
| | NSENELDRDH LGFKIIDNKT YYYDEDSKLV KGLININNSL |
| | FYFDPIESNL |
| | VTGWQTINGK KYYFDINTGA ASTSYKIING KHFYFNNNGV |
| | MQLGVFKGPD |
| | GFEYFAPANT QNNNIEGQAI VYQSKFLTLN GKKYYFDNDS |
| | KAVTGWRIIN |
| | NEKYYFNPNN AIAAVGLQVI DNNKYYFNPD TAIISKGWQT |
| | VNGSRYYFDT |
| | DTAIAFNGYK TIDGKHFYFD SDCVVKIGVF SGSNGFEYFA |
| | PANTYNNNIE |
| | GQAIVYQSKF LTLNGKKYYF DNNSKAVTGW QTIDSKKYYF |
| | NTNTAEAATG |
| | WQTIDGKKYY FNTNTAEAAT GWQTIDGKKY YFNTNTSIAS |
| | TGYTIINGKY |
| | FYFNTDGIMQ IGVFKVPNGF EYFAPANTHN NNIEGQAILY |
| | QNKFLTLNGK |
| | KYYFGSDSKA ITGWQTIDGK KYYFNPNNAI AATHLCTINN |
| | DKYYFSYDGI |
| | LQNGYITIER NNFYFDANNE SKMVTGVFKG PNGFEYFAPA |
| | NTHNNNIEGQ |
| | AIVYQNKFLT LNGKKYYFDN DSKAVTGWQT IDSKKYYFNL |
| | NTAVAVTGWQ |
| | TIDGEKYYFN LNTAEAATGW QTIDGKRYYF NTNTYIASTG |
| | YTIINGKHFY |
| | FNTDGIMQIG VFKGPDGFEY FAPANTHNNN IEGQAILYQN |
| | KFLTLNGKKY |
| | YFGSDSKAVT GLRTIDGKKY YFNTNTAVAV TGWQTINGKK |
| | YYFNTNTYIA |
| | STGYTIISGK HFYFNTDGIM QIGVFKGPDG FEYFAPANTD |
| | ANNIEGQAIR |
| | YQNRFLYLHD NIYYFGNDSK AATGWATIDG NRYYFEPNTA |
| | MGANGYKTID |
| | NKNFYFRNGL PQIGVFKGPN GFEYFAPANT DANNIDGQAI |
| | RYQNRFLEILL |
| | GKIYYFGNNS KAVTGWQTIN SKVYYFMPDT AMAAAGGLFE |
| | IDGVIYFFGV |
| | DGVKAPGIYG |
| 6 | GLININNSLF YFDPIESNLV TGWQTINGKK YYFDINTGAA STSYKIINGK |
| | HFYFNNNGVM QLGVFKGPDG FEYFAPANTQ NNNIEGQAIV |
| | YQSKFLTLNG |
| | KKYYFDNDSK AVTGWRIINN EKYYFNPNNA IAAVGLQVID |
| | NNKYYFNPDT |
| | AIISKGWQTV NGSRYYFDTD TAIAFNGYKT IDGKHFYFDS |
| | DCVVKIGVFS |
| | GSNGFEYFAP ANTYNNNIEG QAIVYQSKFL TLNGKKYYFD |
| | NNSKAVTGWQ |
| | TIDSKKYYFN TNTAEAATGW QTIDGKKYYF NTNTAEAATG |
| | WQTIDGKKYY |
| | FNTNTSIAST GYTIINGKYF YFNTDGIMQI GVFKVPNGFE |
| | YFAPANTHNN |
| | NIEGQAILYQ NKFLTLNGKK YYFGSDSKAI TGWQTIDGKK |
| | YYFNPNNAIA |
| | ATHLCTINND KYYFSYDGIL QNGYITIERN NFYFDANNES |
| | KMVTGVFKGP |
| | NGFEYFAPAN THNNNIEGQA IVYQNKFLTL NGKKYYFDND |
| | SKAVTGWQTI |
| | DSKKYYFNLN TAVAVTGWQT IDGEKYYFNL NTAEAATGWQ |
| | TIDGKRYYFN |
| | TNTYIASTGY TIINGKHFYF NTDGIMQIGV FKGPDGFEYF |
| | APANTHNNNI |
| | EGQAILYQNK FLTLNGKKYY FGSDSKAVTG LRTIDGKKYY |
| | FNTNTAVAVT |
| | GWQTINGKKY YFNTNTYIAS TGYTIISGKH FYFNTDGIMQ |
| | IGVFKGPDGF |
| | EYFAPANTDA NNIEGQAIRY QNRFLYLHDN IYYFGNDSKA |
| | ATGWATIDGN |
| | RYYFEPNTAM GANGYKTIDN KNFYFRNGLP QIGVFKGPNG |
| | FEYFAPANTD |
| | ANNIDGQAIR YQNRFLHLLG KIYYFGNNSK AVTGWQTINS |
| | KVYYFMPDTA |
| | MAAAGGLFEI DGVIYFFGVD GVKAPGIYG |

1. An immunogenic composition comprising:
(a) inactivated whole cells of one or more strains of C. difficile bacteria, a cell surface extract (CSE) of one or more strains of C. difficile bacteria, or a purified component of a CSE of one or more strains of C. difficile bacteria, and
(b) at least one polypeptide comprising a toxoid or a non-toxic immunogenic polypeptide fragment of a C. difficile Toxin A or Toxin B,
wherein said composition, when administered to a mammalian subject, is effective to elicit production of antibodies recognizing a wild type C. difficile and production of antibodies recognizing at least one C. difficile toxin that confers protection against C. difficile infection and/or reinfection (recurrence).

2. The immunogenic composition of claim 1, which comprises the inactivated whole cells of one or more strains of C. difficile bacteria.

3. The immunogenic composition of claim 1, which comprises the CSE of one or more strains of C. difficile bacteria.

4. The immunogenic composition of claim 1, which comprises the purified component of a CSE of one or more strains of C. difficile bacteria.

5. The immunogenic composition of claim 3, wherein the CSE comprises one or more of CbpA, GroEL, CD3246, CD2381, CD0873, Dif51, Dif130, Dif192, Dif208, Dif208A, Dif232, and CDT.

6. The immunogenic composition of claim 3, wherein the CSE of one or more strains of C. difficile bacteria is from a suspension of C. difficile cells in sodium deoxycholate.

7. The immunogenic composition of claim 4, wherein the purified component of a CSE of one or more strains of C. difficile bacteria is a polysaccharide.

8. The immunogenic composition of claim 7, wherein the polysaccharide is a polymer of hexasaccharide phosphate-repeats.

9. The immunogenic composition of claim 7, wherein the polysaccharide is PS II.

10. The immunogenic composition of claim 3, wherein the composition comprises one or more recombinantly expressed cell surface components.

11. The immunogenic composition of claim 1, wherein the one or more C. difficile strains comprises a C. difficile ribotype selected from the group consisting of C. difficile ribotypes 001, 003, 027, 106, 012, 014, and 036.

12. The immunogenic composition of claim 1, wherein the composition comprises inactivated whole cells of two C. difficile strains, a purified component of CSEs of two C. difficile strains or CSEs of two C. difficile strains.

13. The immunogenic composition of claim 1, wherein the inactivated whole cells are inactivated by heat treatment, UV or gamma radiation, formaldehyde treatment, treatment with antibiotics, or treatment with alcohols.

14. The immunogenic composition of claim 13, wherein the inactivated whole cells are inactivated by treatment with ethanol, isopropyl alcohol, phenol, tricresol, or combinations thereof.

15. The immunogenic composition of claim 13, wherein the inactivated whole cells are inactivated by treatment with β-propiolactone (BPL).

16. The immunogenic composition of claim 13, wherein the inactivated whole cells are inactivated by heating a culture of C. difficile at 65°-80° C. for at least 20 minutes.

17. The immunogenic composition of claim 13, wherein the inactivated whole cells are inactivated by suspending C. difficile cells in formalin.

18. The immunogenic composition of claim 1, wherein component (b) comprises a toxoid of a C. difficile Toxin A and/or a toxoid of a C. difficile Toxin B.

19. The immunogenic composition of claim 1, wherein component (b) comprises a non-toxic polypeptide fragment of C. difficile Toxin A and/or a non-toxic fragment of C. difficile Toxin B.

20. The immunogenic composition of claim 19, wherein the composition comprises a non-toxic polypeptide fragment having at least 80% identity to SEQ ID NO:7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

21. The immunogenic composition of claim 19, wherein the composition comprises a non-toxic polypeptide fragment of C. difficile Toxin A that binds to actoxumab.

22. The immunogenic composition of claim 19, wherein the composition comprises a non-toxic polypeptide fragment of C. difficile Toxin B that binds to bezlotoxumab.

23. The immunogenic composition of claim 1, wherein component (b) comprises at least one CROPs region of C. difficile Toxin A, at least one CROPs region of C. difficile Toxin B, or a combination thereof.

24. The immunogenic composition of claim 23, wherein component (b) comprises a polypeptide the C-terminal 716 amino acids of a C. difficile Toxin B or immunogenic fragment thereof.

25. The immunogenic composition of claim 18, wherein the Toxin A and/or the Toxin B comprises toxins from more than one C. difficile strain.

26. The immunogenic composition of claim 19, wherein the Toxin A polypeptide fragment and/or the Toxin B polypeptide fragment comprises fragments from more than one C. difficile strain.

27. The immunogenic composition of claim 1, wherein component (b) comprises a polypeptide from one or more strains selected from the group consisting of C. difficile ribotypes 003, 027, 106, 001, 012, 014, 036, and 078.

28. The immunogenic composition of claim 27, wherein component (b) comprises a polypeptide from a C. difficile BI/NAP1/027 strain.

29. The immunogenic composition of claim 28, wherein component (b) comprises a polypeptide having the amino acid sequence of SEQ ID NO:3.

30. The immunogenic composition of claim 1, wherein component (a) comprises inactivated whole cells of a C. difficile BI/NAP1/027 strain, and component (b) comprises a non-toxic, immunogenic polypeptide fragment of Toxin A and/or Toxin B from a C. difficile BI/NAP1/027 strain.

31. The immunogenic composition of any one of the preceding claims, further comprising: (c) an adjuvant.

32. The immunogenic composition of claim 31, wherein the adjuvant is selected from the group consisting of alum, mineral oil, vegetable oils, aluminum hydroxide, Freund's incomplete adjuvant, and microparticles or beads of biocompatible matrix materials.

33. The immunogenic composition of claim 31, wherein the adjuvant is alum.

34. A method of making an immunogenic composition effective for eliciting an immune response producing antibodies reactive with one or more strains of C. difficile and producing antibodies reactive with one or more C. difficile toxins, said method comprising the steps:
(1) admixing a first component (a) comprising inactivated whole cells of at least one strain of C. difficile, a cell surface extract (CSE) of at least one strain of C. difficile bacteria, or a purified component of a CSE of at least one strain of C. difficile bacteria in an amount effective to elicit an immune response in a mammalian subject immunized with said first component to produce antibodies reactive with the at least one strain of C. difficile with a second component (b) comprising at least one polypeptide comprising a toxoid or a non-toxic immunogenic polypeptide fragment of a C. difficile Toxin A or Toxin B in an amount effective to elicit an immune response in a mammalian subject immunized with said second component to produce antibodies reactive with at least one of said C. difficile Toxin A or Toxin B; and
(2) formulating said admixture of step (1) for administration to a mammalian subject susceptible to C. difficile infection.

35. A method of eliciting an immune response in a subject against C. difficile, said method comprising administering to said subject an amount of an immunogenic composition comprising: (a) inactivated whole cells of at least one strain of C. difficile, a cell surface extract (CSE) of at least one strain of C. difficile bacteria or a purified component of a CSE of at least one strain of C. difficile bacteria, and (b) at least one polypeptide comprising a toxoid or a non-toxic immunogenic polypeptide fragment of a C. difficile Toxin A or Toxin B.

36. The method of claim 35, which further comprises administering an antibiotic.

37. A method of prophylaxis to prevent C. difficile infection is a subject treated with an antibiotic, which comprises administering to the subject an immunogenic composition comprising: (a) inactivated whole cells of at least one strain of C. difficile, a cell surface extract (CSE) of at least one strain of C. difficile bacteria or a purified component of a CSE of at least one strain of C. difficile bacteria, and (b) at least one polypeptide comprising a toxoid or a non-toxic immunogenic polypeptide fragment of a C. difficile Toxin A or Toxin B.

38. A method of treating an infection in a subject, the method comprising administering to the subject an amount of an antibiotic effective to treat the infection and an amount of an immunogenic composition comprising: (a) inactivated whole cells of at least one strain of C. difficile, a cell surface extract (CSE) of at least one strain of C. difficile bacteria or a purified component of a CSE of at least one strain of C. difficile bacteria, and (b) at least one polypeptide comprising a toxoid or a non-toxic immunogenic polypeptide fragment of a C. difficile Toxin A or Toxin B to prevent reinfection.

39. A method of prophylactically reducing pathogenic symptoms associated with C. difficile infection in a subject, said method comprising administering to said subject a composition comprising (a) inactivated whole cells of at least one strain of C. difficile, a cell surface extract (CSE) of at least one strain of C. difficile bacteria or a purified component of a CSE of at least one strain of C. difficile bacteria, and (b) at least one polypeptide comprising a toxoid or a non-toxic immunogenic polypeptide fragment of a C. difficile Toxin A or Toxin B.

40. The method of claim 39, which further comprises passive immunization.
